(19) United States
(12) Patent Application Publication
(10) Pub. No.: US 2023/0009725 A1
(43) Pub. Date: Jan. 12, 2023

(54) USE OF GENETIC ALGORITHMS TO DETERMINE A MODEL TO IDENTITY SAMPLE PROPERTIES BASED ON RAMAN SPECTRA

(71) Applicant: GENENTECH, INC., South San Francisco, CA (US)

(72) Inventors: Dana Lauren McDaniel, South San Francisco, CA (US); Elvira Kadaub, South San Francisco, CA (US); Bingchuan Wei, South San Francisco, CA (US); Andrew James Maier, South San Francisco, CA (US); Gordon Edward Magill, South San Francisco, CA (US)

(73) Assignee: GENENTECH, INC., South San Francisco, CA (US)

(21) Appl. No.: 17/947,820

(22) Filed: Sep. 19, 2022

Related U.S. Application Data
(63) Continuation of application No. PCT/US2021/025921, filed on Apr. 6, 2021.
(60) Provisional application No. 63/008,196, filed on Apr. 10, 2020.

(51) Int. Cl. G01N 21/65; G06N 3/12; G16C 20/70

(52) U.S. Cl. CPC G01N 21/65; G06N 3/126; G16C 20/70

(57) ABSTRACT

Techniques are disclosed for using a genetic algorithm to identify a processing pipeline that transforms spectra into a form usable to generate predicted characteristics of corresponding samples. The genetic algorithm is used to generate and evaluate multiple candidate solutions specifying various pre-processing and machine-learning-processing configurations. The processing pipeline is defined based on the candidate solutions.

### CROSS-REFERENCES TO RELATED APPLICATIONS

The present application is a continuation of International Application Number PCT/US2021/025921, filed on Apr. 6, 2021, which claims the benefit of and priority to U.S. Provisional Application No. 63/008,196, filed Apr. 10, 2020. The entire contents of each of these applications is incorporated herein by reference in its entirety for all purposes.

### BACKGROUND

Quality control techniques are frequently implemented to monitor attributes for the development of new drugs and research samples in order to ensure uniformity across the development and production process. Even slight variations in the production or molecular structure of a new drug or research sample can lead to discrepancies in both treatment and experimental outcomes. For this reason, it is important to maintain a consistent set of attributes and overall measure of quality for any given sample of a biopharmaceutical drug or compound.

Comparison of many properties of biopharmaceutical drugs and/or materials to reference metrics can indicate the quality of a sample. For example, the pH of a sample can be measured to indicate whether a compound or drug has an expected acidic or basic nature. As another example, the osmolality of a sample can be measured to indicate whether a concentration of solute within a solution for the sample matches a target osmolality associated with a high-quality reference sample. The measurement of such properties may disclose the purity or stability of a molecule or compound, and the accuracy and/or consistency of mass production of a biopharmaceutical drug before its distribution to subjects.

Current techniques for data processing and model determination take significant computational and time resources, as trained experts in the field manually choose a set of techniques for analyzing a sample and define target values and/or ranges for sample attributes.

### SUMMARY

The use of an automated data processing pipeline utilizing spectra data and tandem machine-learning models to quantify characteristics of a sample may utilize less resources (e.g., decreased computing time and/or decreased manual time designing an optimal machine-learning model), increase the accuracy of quality prediction, and reduce user-to-user variability in processing techniques.

Some embodiments of the present disclosure include a computer-implemented method. A data set can be accessed. The data set can include a set of first data elements, each of which includes a spectrum corresponding to a sample. The spectrum may have been generated using spectroscopy, such that it was based on an interaction between a sample and energy from an energy source. For example, the spectrum may have been generated using Raman spectroscopy, infrared spectroscopy, mass spectrometry, liquid chromatography, or nuclear magnetic resonance (NMR) spectroscopy.

The data set can include a set of corresponding labels, each of which indicates a known characteristic of the associated sample. A population of candidate solutions is initialized. Each of the population of candidate solutions is defined by a set of properties that indicate whether a particular type of pre-processing is to be performed; a parameter of a pre-processing technique is to be used; which type of machine-learning model is to be used; and/or which machine-learning hyperparameter(s) to apply.

A single solution can be determined by filtering (equally, selecting from among) the population of candidate solutions. The filtering can include determining, for each of the population of candidate solutions and for each of at least some of the input data elements of the data set, a predicted sample characteristic by processing the spectrum of a data element in accordance with the set of properties. The filtering can further include selecting an incomplete subset of the population of candidate solutions based on a fitness metrics. One or more additional generation iterations can be performed by updating the population of candidate solutions to include a next-generation population of solutions identified using the selected incomplete subset of the population of candidate solutions and one or more genetic operators. The one or more genetic operators may include a selection technique(s) and/or a mutation rate. The filtering of the population of candidate solutions using the updated population of candidate solutions is repeated until a termination condition is satisfied (e.g., having completed processing for a predetermined number of generations or a having detected that a solution with an estimated error below a predefined threshold has been determined).

After the termination condition is satisfied, a processing pipeline is defined based upon the set of properties of a particular candidate solution in the incomplete subset selected during a final generation. Thus, the processing pipeline can include configuration information for pre-processing and/or machine-learning processing that is based at least in part on the set of properties. In some instances, another spectrum corresponding to another sample may be accessed. A predicted characteristic of the other sample is generated by processing (e.g., which can include pre-processing and/or processing performed by a machine-learning model) the other spectrum in accordance with the configuration information from the processing pipeline. The predicted characteristic of the other sample is output (e.g., presented or transmitted to a user device).

In some embodiments, a system is provided that includes one or more data processors and a non-transitory computer readable storage medium containing instructions which, when executed on the one or more data processors, cause the one or more data processors to perform part or all of one or more methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 shows an exemplary interaction system for using a genetic algorithm to facilitate quality-control processing of samples, in accordance with some embodiments of the invention.

FIG. 2 illustrates an example of a feature-selection controller 112 that selects features for use in estimating or predicting sample characteristics, in accordance with some embodiments of the invention

FIG. 3 shows an exemplary process 300 for using a genetic algorithm to facilitate quality-control processing of samples, in accordance with some embodiments of the invention.

FIG. 4 shows an exemplary population of candidate solutions and corresponding properties for each candidate solution of the population of candidate solutions for a single generation, in accordance with some embodiments of the invention.

FIG. 5A shows exemplary comparisons between the measured label values of lactate concentration and the predicted label values of lactate concentration generated by an exemplary first-generation candidate processing pipeline, in accordance with some embodiments of the invention.

FIG. 5B shows exemplary comparisons between the measured label values of lactate concentration and the predicted label values of lactate concentration generated by a selected last-generation processing pipeline, in accordance with some embodiments of the invention.

FIG. 6A shows exemplary comparisons between the measured label values of glucose concentration and the predicted label values of glucose concentration generated by an exemplary first-generation candidate processing pipeline, in accordance with some embodiments of the invention.

FIG. 6B shows exemplary comparisons between the measured label values of glucose concentration and the predicted label values of glucose concentration generated by a selected last-generation processing pipeline, in accordance with some embodiments of the invention.

FIG. 7A shows exemplary comparisons between the measured label values of pH and the predicted label values of pH generated by an exemplary first-generation candidate processing pipeline, in accordance with some embodiments of the invention.

FIG. 7B shows exemplary comparisons between the measured label values of pH and the predicted label values of pH generated by a selected last-generation processing pipeline, in accordance with some embodiments of the invention.

FIG. 8A shows exemplary comparisons between the measured label values of osmolality and the predicted label values of osmolality generated by an exemplary first-generation candidate processing pipeline, in accordance with some embodiments of the invention.

FIG. 8B shows exemplary comparisons between the measured label values of osmolality and the predicted label values of osmolality generated by a selected last-generation processing pipeline, in accordance with some embodiments of the invention.

FIG. 9A shows exemplary comparisons between the measured label values of antibody oxidation and the predicted label values of antibody oxidation generated by an exemplary first-generation candidate processing pipeline, in accordance with some embodiments of the invention.

FIG. 9B shows exemplary comparisons between the measured label values of antibody oxidation and the predicted label values of antibody oxidation generated by a selected last-generation processing pipeline, in accordance with some embodiments of the invention.

FIG. 10A shows exemplary comparisons between the measured label values of Glycan G0F-N and the predicted label values of Glycan G0F-N generated by an exemplary first-generation candidate processing pipeline, in accordance with some embodiments of the invention.

FIG. 10B shows exemplary comparisons between the measured label values of Glycan GOF-N and the predicted label values of Glycan GOF-N generated by a selected last-generation processing pipeline, in accordance with some embodiments of the invention.

FIG. 11A shows exemplary comparisons between the measured label values of a sum of HMWF and the predicted label values of the sum of HMWF generated by an exemplary first-generation candidate processing pipeline, in accordance with some embodiments of the invention.

FIG. 11B shows exemplary comparisons between the measured label values of a sum of HMWF and the predicted label values of the sum of HMWF generated by a selected last-generation processing pipeline, in accordance with some embodiments of the invention.

FIG. 12A shows exemplary comparisons between the measured label values of bispecific assembly and the predicted label values of bispecific assembly generated by an exemplary first-generation candidate processing pipeline, in accordance with some embodiments of the invention.

FIG. 12B shows exemplary comparisons between the measured label values of bispecific assembly and the predicted label values of bispecific assembly generated by a selected last-generation processing pipeline, in accordance with some embodiments of the invention.

FIG. 13A shows exemplary comparisons between the measured label values of an abundance of viable cells and the predicted label values of the abundance of viable cells generated by an exemplary first-generation candidate processing pipeline, in accordance with some embodiments of the invention.

FIG. 13B shows exemplary comparisons between the measured label values of an abundance of viable cells and the predicted label values of the abundance of viable cells generated by a selected last-generation processing pipeline, in accordance with some embodiments of the invention.

FIG. 14A shows exemplary comparisons between the measured label values of an abundance of dead cells and the predicted label values of the abundance of dead cells generated by an exemplary first-generation candidate processing pipeline, in accordance with some embodiments of the invention.

FIG. 14B shows exemplary comparisons between the measured label values of an abundance of dead cells and the predicted label values of the abundance of dead cells generated by a selected last-generation processing pipeline, in accordance with some embodiments of the invention.

FIG. 15A shows exemplary comparisons between the measured label values of a residual moisture content and the predicted label values of the residual moisture content generated by an exemplary first-generation candidate processing pipeline, in accordance with some embodiments of the invention.

FIG. 15B shows exemplary comparisons between the measured label values of a residual moisture content and the predicted label values of the residual moisture content generated by a selected last-generation processing pipeline, in accordance with some embodiments of the invention.

FIG. 16A shows an exemplary set of spectra prior to spectral preprocessing, in accordance with some embodiments of the invention.

FIG. 16B shows the exemplary set of spectra following spectral preprocessing performed in accordance with a processing pipeline defined using pH labels and a genetic algorithm, in accordance with some embodiments of the invention.

FIG. 17A shows an exemplary set of spectra prior to spectral preprocessing, in accordance with some embodiments of the invention.

FIG. 17B shows the exemplary set of spectra following spectral preprocessing performed in accordance with a processing pipeline defined using antibody oxidation labels and a genetic algorithm, in accordance with some embodiments of the invention.

FIG. 18A shows an exemplary set of spectra prior to spectral preprocessing, in accordance with some embodiments of the invention.

FIG. 18B shows the exemplary set of spectra following spectral preprocessing performed in accordance with a processing pipeline defined using bispecific assembly labels and a genetic algorithm, in accordance with some embodiments of the invention.

FIG. 19A shows an exemplary set of spectra prior to spectral preprocessing, in accordance with some embodiments of the invention.

FIG. 19B shows the exemplary set of spectra following spectral preprocessing performed in accordance with a processing pipeline defined using labels for an abundance of viable cells and a genetic algorithm, in accordance with some embodiments of the invention.

FIG. 20A shows an exemplary set of spectra prior to spectral preprocessing, in accordance with some embodiments of the invention.

FIG. 20B shows the exemplary set of spectra following spectral preprocessing performed in accordance with a processing pipeline defined using labels for an abundance of dead cells and a genetic algorithm, in accordance with some embodiments of the invention.

FIG. 21A shows an exemplary set of spectra prior to spectral preprocessing, in accordance with some embodiments of the invention.

FIG. 21B shows the exemplary set of spectra following spectral preprocessing performed in accordance with a processing pipeline defined using labels for a residual moisture content and a genetic algorithm, in accordance with some embodiments of the invention.

FIG. 22A shows the exemplary set of spectra before spectra preprocessing, in accordance with some embodiments of the invention.

FIG. 22B shows an exemplary set of a spectra following a feature-selection process in accordance with a processing stage of a processing pipeline, in accordance with some embodiments of the invention.

FIG. 23 shows an exemplary set of iterations of a feature-selection process to identify a particular reduced set of features for estimating a characteristic of a sample, in accordance with some embodiments of the invention.

FIGS. 24A-24D illustrate graphs that correspond to the exemplary set of iterations of FIG. 23, in accordance with some embodiments of the invention.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

A genetic algorithm can be used to define a data processing pipeline that can be used to estimate a characteristic of a sample. The sample may be (for example) a biopharmaceutical product or drug and/or may include a small-molecule active ingredient and/or large-molecule active ingredient. The characteristic can include (for example) a concentration of one or more small-molecule analytes, identification of a solvent, characterization of a solvent, prevalence of one or more protein variants, pH, osmolality, protein homogeneity, protein structure (e.g., a protein higher-order structure), or large molecule impurities (e.g., a high concentration of host-cell proteins) of the sample. The processing pipeline can include processing a spectrum representing a result of an interaction between energy from an energy source and the sample. The spectrum may be processed by using a machine-learning model (e.g., a partial least squares model, random forest model or support vector machine model). The processing pipeline may further include pre-processing the spectrum (e.g., to remove a baseline, scale the spectrum and/or smooth the spectrum).

The genetic algorithm can be used to determine a set of properties of the processing pipeline that include whether a particular type of pre-processing is to be performed; a parameter of a pre-processing to be performed; which type of machine-learning model is to be used; and/or which machine-learning hyperparameter(s) to apply. For example, a type of pre-processing may include baseline removal (e.g., a linear or nonlinear subtraction of signal data to reduce noise and/or remove fluorescent or other spectral interference within a spectrum), scaling (e.g., proportionally transforming spectral data in order to enable comparisons from different contexts), outlier identification and/or removal, and/or smoothing (e.g., a reduction of remaining fluctuations within spectral data). In some instances, a parameter may indicate whether a more specific type of pre-processing is to be performed or which specific type of pre-processing is to be performed. For example, a parameter may include a selection of one of the following techniques to use for baseline removal: asymmetric least squares, adaptive iteratively reweighted Penalized Least Squares, Fully Automatic Baseline Correction, the Kajfosz-Kwiatek method. A parameter of pre-processing to be performed may include (for example) a decay value, a weight, a penalty, or a filter. A parameter of pre-processing to be performed may include (for example) a type of scaling such as row-wise and/or column-wise unit variance (e.g., with the unit variance scaling each variable (column) as (value-mean)/standard deviation). A type of machine-learning model may include (for example) a random forest model, a support vector model, a regression model, a neural network (e.g., of a particular type, such as a recurrent neural network, a deep neural network, and/or the like) or a model based upon a combination of more than one common machine-learning models. A machine-learning hyperparameter may include (for example) a learning rate, a number of generations, and a number of trees and/or leaves, such that the hyperparameters are based upon the type of machine-learning model that is chosen. As an example, a random forest model may include a hyperparameter defining a number of trees, while a linear regression model would not necessarily include a hyperparameter for the number of trees.

The genetic algorithm can determine the set of properties by iteratively defining and evaluating a set of candidate solutions. Each candidate solution can include particular properties that define a type of pre-processing to be performed (and/or one or more parameters thereof) and/or a type of machine-learning model to be used in processing of a (raw or pre-processed) spectrum (and/or one or more hyperparameters thereof). More specifically, each iteration can be referred to as a generation iteration and can include assessment of a population of candidate solutions. The assessment can include generating, for each candidate solution in the population, a fitness metric that indicates how well the processing pipeline configured with properties associated with the candidate solution performed in relation to the known characteristic (e.g., an accuracy metric, error metric, sensitivity metric, etc.). For example, the fitness metric may be or include a mean square error (MAE), a root mean square error (RMSE), or a log-hyperbolic-cosine-error (log(cosh)). An incomplete subset of the population of candidate solutions can then be selected based on the fitness metrics (e.g., so as to identify a particular number of candidate solutions associated with the highest fitness metrics in the population or to identify each candidate solution in the population that is associated with a fitness metric above a predetermined threshold). In some instances, the population of candidate solutions are ranked by their corresponding fitness metric. As such, when determining the incomplete subset of candidate solutions, a genetic algorithm may select several candidate solutions with the highest ranking in relation to the other candidate solutions within the population. The subset of candidate solutions may then be included within a new population of candidate solutions for a next generation.

A new population of candidate solutions for a next generation may consist of the selected candidate solutions of the determined subset along with a new set of candidate solutions generated by the genetic algorithm using a set of genetic operators (e.g., a mutation rate). The genetic operators may be configured to generate new candidate solutions based upon commonly used methods for measuring a characteristic (as opposed to random generation). Furthermore, for each new generation, the number of candidate solutions within a population may stay constant. For example, if the genetic algorithm selects 2 candidate solutions from a total population of 20 candidate solutions to proceed to a next generation, the genetic algorithm will generate 18 additional candidate solutions for a total of 20 candidate solutions within the next generation. The next generation iteration can determine a ranking for the new population of candidate solutions and select a new subset of candidate solutions.

Upon completion of a final generation iteration, the genetic algorithm can identify a single solution from the incomplete subset of the population of candidate solutions. In some instances, the incomplete subset has a size of a single solution, and thus, the identified single solution can be that of the incomplete subset. In some instances, the incomplete subset includes multiple solutions, and the single solution may be identified by (for example) selecting a solution from the multiple solutions that is associated with a highest fitness metric.

The single solution can be used to define the processing pipeline, which, in turn, can transform individual spectra to a predicted label corresponding to a predicted sample characteristic. The processing pipeline can process the set of input spectra by potentially performing a pre-processing configured in accordance with a solution's set of properties and performing processing using a machine-learning model configured in accordance with at least some of a solution's set of properties. The processing pipeline can further or additionally process the set of input spectra by processing each spectrum in the set of input spectra (e.g., and/or a pre-processed version thereof) using a machine-learning model selected and/or at least partly configured in accordance with another at least some of the solution's set of properties. The machine-learning model may further be configured in accordance with one or more parameters and/or variables determined and/or learned using a (for example) a training dataset.

In some instances, the processing pipeline is augmented with one or more additional processing steps that are performed before estimating a characteristic of the sample (e.g., before processing the input spectra with a machine-learning model, etc.). For instance, a feature-selection process may be performed to reduce the quantity of features processed by the machine-learning model. A computing device, executing a feature-selection process, represents the input spectra as a set of wavenumbers (e.g., spatial frequency of a wave) with each wavenumber including a corresponding intensity (e.g., a feature). The computing device then selects from the intensities, one or more intensities at a corresponding one or more wavenumbers for use in predicting the characteristic of the input sample.

For example, the computing device can analyze the set of wavenumbers using a regression algorithm (e.g., such as a using partial least squares, or the like) to assign a rank for each wavenumber (e.g., based on relative ordering of the weights of the partial least squares regression). The set of wavenumbers may be sorted according to the rank assigned to each wavenumber. The computing device then defines subsets of wavenumbers with a first subset including each wavenumber (e.g., the full set of wavenumbers) and each subsequent subset excluding one or more wavenumbers from the previous subset (e.g., the lowest ranking wavenumbers, the highest ranking wavenumbers, random wavenumbers, or the like).

The computing device performs an iterative subset analysis that derives a score for each subset to determine the subset that is to be used to estimate the characteristic of the sample. Each score represents a degree to which processing spectra (in accordance with a processing pipeline) that include intensities for wavenumbers in the subset accurately predict a sample characteristic. A test (e.g., hold-out) or validation dataset can be used to characterize performance characteristics (e.g., precision, recall, accuracy, etc.)

During the first iteration, the computing device derives a baseline score (e.g., using a cross-validation analysis) from a test dataset or a validation dataset using spectra that correspond to the subset that includes the set of wavenumbers. That is, full spectra are processed using a defined processing pipeline to predict sample characteristics, and the predicted sample characteristics are compared to true sample characteristics to generate the baseline score. The baseline score can be used as a reference data point to predict an effect that removing (from spectra) intensities at given wavenumbers may have on the accuracy of the machine-learning model to estimate the characteristic of the sample.

During the second iteration, a score is derived for the next subset. This subset includes the wavenumbers from the first iteration (e.g., the set of wavenumbers) with one or more wavenumbers being removed from the set of wavenumbers based on rank (e.g., such as the lowest ranking wavenumbers, highest ranking wavenumbers, random sampling, or the like). In some instances, the computing device may remove the x percent of wavenumbers based on rank (e.g., 5%, 10%, etc.) from wavenumbers present in a previous iteration, potentially rounding up. In other instances, the computing device may remove a predetermined quantity of the wavenumbers. The percentage of wavenumbers or the predetermined quantity that are removed may be configurable (e.g., by user input, by the machine-learning model, hardcoded, etc.).

The computing device then compares the score derived during the second iteration to the baseline score. If the score for this iteration is higher than the baseline score (e.g., indicating that the reduction in wavenumbers improves the estimation of the characteristic), then the score for this iteration becomes the new baseline score and the process continues to the next iteration. If the score for this iteration is not higher than the baseline score, then the process simply continues without updating the baseline score.

During the next iteration, a score is derived for the next subset. This subset includes the wavenumbers from the subset of the second iteration with the next lowest ranking wavenumbers removed. The score may be compared to the baseline score to determine if the score is to be the new baseline score.

After the iterative subset analysis has ended, a determination is made as to which iteration is associated with score that is within a threshold deviation from the baseline score. Specifically, the computing device identifies the iteration in which the score associated with that iteration is closest or equal to (but not exceeding) a threshold deviation from the baseline score. By selecting the spectra (e.g., that correspond to the selected wavenumbers) used to predict the characteristic, the accuracy of the prediction may be impacted. For example, selecting a small portion of the spectra reduces information that may contribute the prediction (e.g., lowering the accuracy of the prediction). The threshold deviation enables selection of a reduced spectra for predicting the characteristic while ensuring the accuracy of the resulting prediction. In one example, if the baseline score is 0.892 and the threshold is 0.020, the iteration having a score that is closest to or equal to 0.872 will be selected. Alternatively, the computing device identifies the iteration in which the score associated with that iteration is closest to the threshold deviation from the baseline score. The computing device selects the intensities (e.g., features) of the wavenumbers from the subset of the identified iteration to be input features for the machine-learning model (e.g., used to estimate the characteristic of the sample).

The computing device may execute the feature-selection process near the end of the processing pipeline, such as before estimating the characteristic of the sample (e.g., using the machine-learning model, or the like). Alternatively, the feature-selection process may be included and/or configured by the genetic algorithm. In this instance, the genetic algorithm can define one or more candidate solutions that include the feature-selection process. The genetic algorithm then determines whether feature selection is to be performed during a stage in the processing pipeline (e.g., through evaluation of the candidate solutions that do or do not include the feature-selection process) and one or more parameters of the feature-selection process such as the quantity of iterations, the score, quantity of features to be removed during each iteration (e.g., percentage, quantity, etc.), or the like.

Subsequent estimations of the characteristic for a new set of samples can utilize the processing pipeline in order to estimate a characteristic and a resulting measure of quality for each of the new set of samples. In the event that estimation of a different characteristic of interest is desired for a set of samples, the genetic algorithm can repeat the above technique of determining another solution in order to generate another processing pipeline for the different characteristic of interest.

A processing pipeline, defined using a genetic algorithm, then receives an input spectrum associated with a particular sample and outputs an estimated characteristics of the particular sample. It will be appreciated that, after the processing pipeline is defined, it may be implemented without further involving and/or executing the genetic algorithm. The estimation of the sample characteristic(s) can be used in a quality-control process to determine whether to release a given sample or batch of samples for distribution for potential administration or actual administration to one or more subjects. For example, the quality-control process may include evaluating a quality-control condition using an estimated characteristic of a sample. The quality-control condition may be configured to be satisfied (for example) when an estimated characteristic matches a particular value, is within a predefined range, is less than an upper threshold and/or is lower than a lower threshold. In some instances, a quality-control condition is assessed at a batch level, which can include generating a statistic (e.g., mean, median, standard deviation, range, variance, etc.) based on a distribution of estimated characteristics for the batch of samples and determining whether the statistic is (for example) below a predefined batch upper threshold and/or above a predefined batch lower threshold. When it is determined that the quality-control condition is satisfied, the sample(s) may be marked or approved for distribution (e.g., shipment). When it is determined that the quality-control condition is not satisfied, such distribution may be prevented (e.g., by marking the sample(s) as being unapproved and/or pulling the sample(s) from a production line).

In some instances where the quality-control condition is not satisfied, the discrepancies within the estimated characteristics for the batch of samples may determine a dynamic adjustment within a production and/or manufacture process for the generation of future samples (e.g., which may include a bioprocess for generating samples including large molecules). For example, a production process may be modified to include an addition or a removal of an ingredient of a sample in response to an estimated characteristic for the ingredient being too low or too high, respectively. In another example, a production process may be modified to add, change or remove one or more processing steps (e.g., to add an additional purification of a sample, change a temperature of a processing step, etc.) in response to an estimated characteristic not satisfying the quality-control condition. In some instances, a result of an assessment of a quality-control condition influences whether a manufacture process is initiated, re-initiated and/or terminated. For example, a manufacture process may be periodically paused to evaluate select samples and determine whether the quality-control condition is satisfied. If so, the process can be re-initiated. If not, one or more aspects of the process may be modified.

### II. Exemplary Interaction System

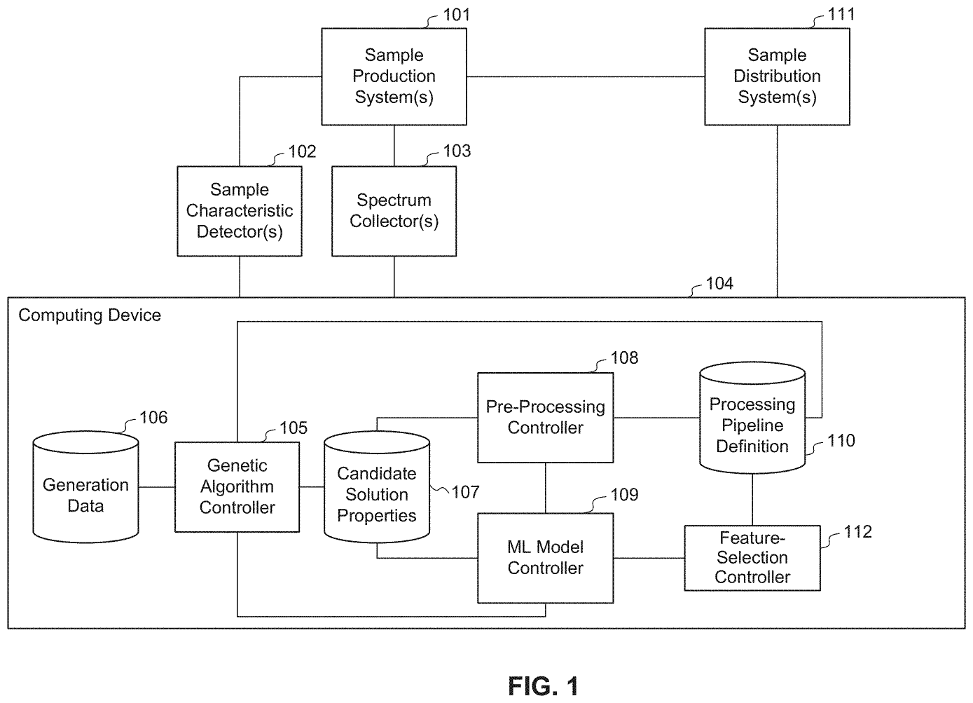

FIG. 1 shows an exemplary interaction system for using a genetic algorithm to facilitate quality-control processing of samples, in accordance with some embodiments of the invention. One or more sample production systems 101 produce a set of samples. Each sample of the set of samples may include (for example) a pharmaceutical and/or drug sample to be used (for example) for a diagnostic and/or treatment purpose. Each sample of the set of samples may include (for example) one or more active ingredients that includes small molecules and/or large molecules and one or more inactive ingredients. Sample production system(s) 101 can include a laboratory.

At least some of the samples are processed via one or more sample characteristic detectors 102, which identify one or more characteristics of the sample. The one or more characteristics of the sample include a characteristic of an active ingredient, a characteristic of an inactive ingredient and/or a characteristic of the sample as a whole. Exemplary characteristics for a small molecule include (but are not limited to) an active ingredient concentration, a lactose concentration, or a microcrystalline cellulose concentration. An exemplary characteristic for a large molecule can include (but are not limited to) any impurities (e.g., an abundance of an unreacted element, a concentration of host cell proteins, and/or a concentration of any residual undesired proteins) within the large molecule. The characteristic can additionally include a numeric or categorical characteristic. The at least some of the samples that are processed via one or more sample characteristic detectors 102 can include (for example) samples that are to be represented in a training, validation or testing set.

A spectrum collector 103 can process each sample of the set of samples to generate a spectrum. A spectrum includes an intensity for each of multiple wavenumbers. The process can include energizing each sample with energy from an energy source and detecting a subsequent spectra. The energy source may include (for example) a light source that emits light energy or a physical-energy source that emits physical energy. In some instances, the spectrum is collected in a non-destructive manner, such that the sample is not destroyed and/or degraded as a result of the spectrum collection. The spectrum can be obtained by performing (for example) Raman spectroscopy, infrared spectroscopy, mass spectrometry, liquid chromatography, or NMR spectroscopy. Exemplary types of infrared spectroscopy can include near infrared (NIR), mid infrared (MIRA), thermal infrared (TIR) or Fourier-transform infrared (FTIR) spectroscopy.

In some instances, multiple spectra may be collected using a single sample. Thus, each of the multiple spectra can be associated with a same one or more sample characteristics, given that they pertain to the same sample. The multiple spectra can be referred to as replicate spectra. Differences between the spectra may be due to (for example) slight shifting of a sample container across scans and/or spectra-recording machine inconsistencies. Differences across the same-spectra samples can include (for example) differences in peak height, peak width, peak location and/or jitter. The differences may be relatively small, though they may nonetheless impact training and/or a quality of a processing pipeline. An Extended Multiplicative Scatter Correction algorithm can be used to process the replicate spectra to identify the idiosyncratic error. Individual spectra can be preprocessed to correct for the idiosyncratic error using linear correction, as described in Marterns, H. & Stark, E. (1991). Extended multiplicative signal correction and spectral interference subtraction: new preprocessing methods for near infrared spectroscopy. Journal of Pharmaceutical and Biomedical Analysis, 9(8), 625-635, which is hereby incorporated by reference in its entirety for all purposes. A higher-order polynomial can be used with fitting and/or correcting a replicate spectrum against an arbitrarily selected “baseline” replicate scan.

The spectra and detected characteristics are transmitted to a computing device 104. Computing device 104 is configured to use a genetic algorithm to identify a processing pipeline that transforms a spectrum to a characteristic of interest and to then implement the processing pipeline.

More specifically, upon identifying a new training instance (e.g., associated with a particular combination of a type of sample and characteristic of interest), a genetic algorithm controller 105 initiates processing of a first generation. Each generation be associated with a population of candidate solutions—each of which are associated with a set of candidate solution properties. Each property of the set of candidate solution properties can specify a characteristic of a pre-processing or machine-learning processing to be performed. Definitions as to which properties are to be identified may be set by a client and/or developer. Any constraints on the properties (e.g., identifying an upper bound, a lower bound, a universe of options from which a property is to be selected, etc.) may further be set by a client and/or developer. In some instances, the genetic algorithm controller 105 may also optimize constraints on the properties in order to identify an upper bound and an lower bound with no need for manual configuration by the client and/or developer. Each of one or more first other properties may be fixed (e.g., and set by a client and/or developer), and each of one or more second other properties may be identified as ones to be learned upon having a processing pipeline defined.

The sets of candidate solution properties associated with the first generation may be selected randomly, manually (e.g., as defined by a client or developer), or according to a pseudo-random selection process. In some instances, the sets of candidate solution properties are selected in accordance with a technique designed to promote selection of properties that cover (or are likely to cover) a value space to at least a defined degree and/or are likely to differ from each other to a defined degree. The selection may further be performed in accordance with one or more biases applied to one or more properties. In some instances, biases are set to zero for a first generation.

Generation data stored in a generation data store 106 identifies a current generation, any biases applied to selection of the candidate solution properties, and/or a number of candidate solutions included in the current generation (which may be equal to a predefined number set by a client and/or developer). Candidate solution properties are stored in a candidate solution properties data store 107 along with associations that tie each set of candidate solution properties to an identifier of the candidate solution.

For each candidate solution, a pre-processing controller 108 configures pre-processing and a machine-learning (ML) model controller 109 configures a machine-learning model in accordance with the candidate solution properties of the candidate solution. Such configurations may include configuring code so as to either have particular types of pre-processing (e.g., baseline removal, scaling, filtering) performed or not; implement a particular technique to use for a type of pre-processing; implement a particular type of machine-learning model; set particular variables for a pre-processing techniques and/or set particular variables (e.g., that are not to be learned) for a machine-learning model. A candidate processing pipeline is then defined to include the configured pre-processing machine-learning model. A processing pipeline definition data store 110 stores the candidate processing pipeline in association with an identifier of the candidate solution.

Pre-processing controller 108 and machine-learning model controller 109 further uses a training data set (that includes multiple spectra and multiple known measurements of a sample characteristic) to determine any data-dependent values (e.g., to learn parameters for a machine-learning value). Other spectra in a validation or testing data set are then processed using the processing pipeline and any data-dependent values to generate estimated sample characteristics. The estimated sample characteristics is compared to known sample characteristics from the validation or testing data set to generate a fitness metric value for various fitness metrics (e.g., coefficient of determination, square-root of mean squared error, cross entropy, etc.) for the candidate solution.

A data set that includes sample characteristics and spectra corresponding to a set of samples is partitioned into multiple subsets (including a training subset, validation subset and/or testing subset). The partitioning may be performed a single time for the entire data set or may be performed two or more times. For example, the data set may be partitioned separately for each generation evaluated using the genetic algorithm; multiple times with respect to processing a single candidate solution during a single generation (e.g., for k-fold validation analyses); etc.

It will be appreciated that multiple data observations may be collected for a given sample. To illustrate, a sample characteristic and a spectrum may have been collected 100 times for a given sample. However, those 100 observations need not have been independent. Rather, they may pertain to replicated observations. For example, the observations may include 10 replicate observations for each of 10 different lots produced for a given sample. In these instances, one approach is to consider the 100 observations as being sufficiently independent to (for example) randomly or pseudo-randomly partition the observations into subsets (e.g., to pseudo-randomly select 20 observations for testing and use the remaining 80 observations for training). Another approach is to instead partition the lots and group the observations within the lots (e.g., to pseudo-randomly select 2 lots for testing and then use the 20 observations associated with those 2 lots for testing, while using the remaining observations for training). This latter approach may improve training and result in test metrics that more accurately predict how the processing would perform with an independent data set.

In some instances, computing device 104 may analyze spectra of a dataset (the subsets and/or lots) to determine if a portion of the spectra (e.g., intensities of one or more wavenumbers, one or more spectra within the spectra, etc.) is an outlier relative to the remaining portions of the spectra. If the portion of the spectra is determined to be an outlier (e.g., deviating from other portions of the spectra by more than a threshold amount), then the spectra (or a portion thereof) may be discarded (or otherwise not used to define the processing pipeline). Outlier detection may also be performed during execution of the processing pipeline to derive a confidence of the accuracy of a estimation or prediction of characteristics of a sample For example, outlier detection can be performed by comparing predictions resulting from the processing pipeline to other predictions by the processing pipeline.

The outlier detection can include performing a principle component analysis (PCA). Specifically, multiple spectra are analyzed to determine a set of principal components. Each of one or more spectra (that may have been in the multiple spectra used to determine the principal components or may be a different spectrum) can then be projected (or recast) along the principal components to generate a transformed representation of the spectrum. For each of the one or more spectra, a distance metric can be calculated based on a distance that separates the transformed representation of the spectrum and a transformed representation of each of one or more other spectra. If the distance metric is larger than a threshold, then the spectrum can be categorized as an outlier.

In some instances, the current input spectra may be discarded and a new input spectra may be obtained for use in defining a processing pipeline. In other instances, the outlier detection may include identifying one or more wavenumbers or one or more spectra within the input spectra that are outliers and filtering the one or more wavenumbers or the one or more spectra (respectively) from the input spectra. The remaining spectra in the input spectra will be used to define the processing pipeline.

Genetic algorithm controller 105 then updates generation data store 106 to associate each candidate-solution identifier with the fitness metric. It will be appreciated that candidate solutions may be evaluated in parallel or iteratively. When a fitness metric has been determined for each candidate solution in the population, genetic algorithm controller 105 determines whether to perform another generation iteration. For example, another generation iteration can be performed when a current generation count is below a predefined generation processing quantity (e.g., as defined by a client or developer), when a best fitness metric across the population for the current generation does not exceed a predefined threshold (e.g., when a lowest error is higher than a given error threshold or when a highest R² value is lower than an R² threshold), or when a best fitness metric across the population for the current generation has not improved by at least a predefined amount relative to a best fitness metric across a population for a previous generation.

When another generation iteration is to be performed, genetic algorithm controller 105 causes a generation count stored in generation data store 106 to increment and identifies new sets of candidate solution properties (with each set being associated with a new candidate solution). The new sets of candidate solution properties are determined based on the previous set of candidate solution properties and corresponding fitness metrics. For example, the selection of the new sets of candidate solution properties can be biased towards properties associated with previous candidate solutions having relatively high fitness metrics and biased against properties associated with previous candidate solution properties having relatively low fitness metrics. Evolutionary selection in a candidate population is adjusted to different scenarios by modifying a mutation rate(s). The mutation rate(s) includes a randomized or pseudo-randomized permutation of preprocessing techniques and machine-learning parameters. The new candidate solutions are processed as were the first-generation candidate solutions, and the generations are iteratively created and assessed until it is determined that another generation iteration is not to be performed.

If another generation is not to be performed, a single candidate solution is selected. The single candidate solution is (for example) the candidate solution associated with the best fitness metric across candidate solutions from the last generation and/or from all generations.

The processing pipeline of the single candidate solution can be augmented with one or more additional processing stages. For example, the processing pipeline can be augmented using feature-selection controller 112 to select, from an input spectra at a particular stage of the processing pipeline, features to be used to estimate or predict sample characteristics. Feature-selection controller 112 may be included in computing device 104 (as shown) or as a separate processing device in communication with computing device 104.

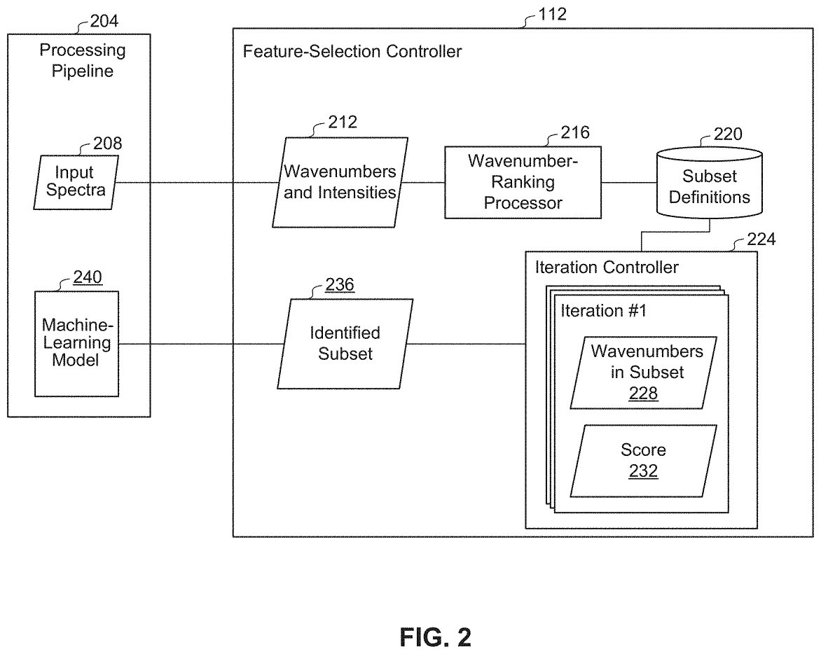

Turning to FIG. 2, which illustrates an example of a feature-selection controller 112 that selects features for use in estimating or predicting sample characteristics, in accordance with some embodiments of the invention. Feature-selection controller 112 may implement a feature-selection process at any stage of the processing pipeline before a stage that generates an estimation or prediction of the sample. For instance feature-selection controller 112 may be operated at a stage prior to operation of a machine-learning model. Input spectra 208 is passed to feature-selection controller 112. Feature-selection controller 112 identifies at 212 a set of wavenumbers in the input spectra and corresponding intensities (e.g., features) at each wavenumber. Feature-selection controller 112 passes the wavenumbers and associated intensities to wavenumber-ranking processor 216, which defines a rank for each wavenumber of the set of wavenumbers.

For example, wavenumber-ranking processor 216 uses a partial least squares (PLS) regression to assign a rank for each wavenumber. PLS outputs a set of components that describe a correlation between a wavenumber and other wavenumbers (e.g., indicative of a degree in which varying the intensity of a wavenumber varies the intensities of other wavenumbers). A rank is assigned to each wavenumber based on a relative ordering of the components of the partial least squares regression.

Feature-selection controller 112 then uses subset definitions 220 to define multiple subsets of the set of wavenumbers based on a quantity of iterations that are to be evaluated for feature selection. In some instances, the number of subsets is equal to the number of iterations to be evaluated. Feature-selection controller 112 defines the subsets by ordering the set of wavenumbers according to rank (e.g., from highest to lowest or vice versa). A first subset includes the full set of wavenumbers. Each subsequent subset includes the wavenumbers from the previous subset excluding a predetermined quantity of the wavenumbers based on rank (e.g., such as the lowest ranking wavenumbers, highest ranking wavenumbers, random selection of wavenumbers, etc.). The predetermined quantity may be a percentage of the quantity of wavenumbers in the set of wavenumbers (potentially rounded up), a percentage of the quantity of wavenumbers in the previous subset, an integer, or the like.

Iteration controller 224 iteratively evaluates each subset of wavenumbers 228 using a cross-validation analysis. The cross-validation analysis is used to generated score 232 for each iteration. Score 232 represents a confidence that estimations or predictions of sample characteristics that are generated using intensities that correspond to wavenumbers in the subset 228 are accurate. Score 232 can be compared to scores of other iterations to determine a relative difference in the confidence of estimations and/or predictions generated using different subsets. The score 232 is derived using a training dataset and a validation dataset that are defined based on the wavenumbers included in subset of wavenumbers 228. The training dataset trains the machine-learning model, which estimates or predicts sample characteristics for the validation dataset (for which ground truth labels are known). A score is derived by comparing the output of processing the validation dataset to the ground truth labels.

Iteration controller 224 outputs an iteration that includes a score that is within a threshold deviation from a baseline score (e.g., the score of the subset that includes the set of wavenumbers). For example, if the threshold deviation is 0.02, iteration controller 224 identifies the iteration having a score that is closest to being 0.02 from the baseline score. The identified subset of wavenumbers 236 includes the subset of wavenumbers of the identified iteration. The intensity at each wavenumber of the identified subset of wavenumbers 236 is then output to machine-learning model 240 in processing pipeline 208 to estimate or predict the sample characteristics.

Returning to FIG. 1, the processing pipeline can be availed to process other spectra (e.g., that are potentially not associated with a known characteristic of the type being estimated by the pipeline) to generate estimated sample characteristics. The processing pipeline that is availed may, but need not, include data-dependent values determined based on training data (e.g., in addition to pre-processing and a machine-learning model configured with the properties associated with the single candidate solution). Availing the processing pipeline may include transmitting code associated with the processing pipeline and/or solution properties of the single candidate solution to another device and/or locally processing other spectra.

The processing pipeline may be used to estimate or predict the characteristics using spectra of other samples, such as samples being prepared for lot release. This includes results that identify, for a given sample, an estimated characteristic that may be locally presented or transmitted to another device. In some instances, a result is only presented or transmitted when a quality-control condition (evaluated using the estimated characteristic) is not satisfied. For example, a result may be conditionally presented when a numeric estimated characteristic is not within a predefined open or closed range or when a numeric estimated characteristic exceeds a particular threshold.

A result may also define an estimated characteristic categorically. Exemplary categories may include labelling a sample as “satisfactory” or “unsatisfactory” based upon whether a quality-control condition is satisfied. In some instances, a category may itself indicate or may be used with one or more categories corresponding to one or more other samples to categorize a lot of samples as satisfactory or unsatisfactory. A lot can correspond to a set of samples manufactured at a single facility during a period of time that may be defined by continuous operation of some or all machines used to manufacture samples and/or during a period of time during which some or all machines used to manufacture samples remain powered on.

Categories may further be defined to identify a characteristic of a sample, particularly in terms of its deficiencies (e.g., a high or low concentration of an active ingredient, a high or low concentration of an inactive ingredient, a high or low pH, etc.). A numeric estimated characteristic may be classified into one of the defined categories based upon predetermined threshold values (e.g., a set of lower or upper bounds for ingredient concentrations, and/or pH, and/or any other suitable sample characteristics) defined by a client and/or developer. An estimated category and/or classification for a characteristic of a sample may be presented or transmitted to another device. As with a numeric estimated characteristic, a result may only be presented when the estimated characteristic has been classified as unsatisfactory or otherwise deficient in some aspect. In some instances, a result may consist of both a numeric estimated characteristic and a categorical estimated characteristic. In such instances, both the numeric estimated characteristic and the categorical estimated characteristic may be presented or transmitted to another device.

An estimated characteristic may be used to determine whether to allow, facilitate, inhibit or prevent a corresponding sample from being distributed by one or more sample distribution systems 111. For example, when the quality-control condition is not satisfied, a communication may be transmitted from computing device 104 to sample distribution system(s) 111 and/or an associated user device that identifies the sample and potentially includes the estimated characteristic and/or an instruction to collect the sample prior from distribution (or remove the sample from an automated sample-distribution processing line). In some instances, sample distribution system 111 and computing device 104 are housed in a same facility. Computing device 104 may be connected to a physical gating mechanism that samples are to traverse prior to distribution. The physical gating mechanism may be configured to selectively pass samples for which the quality-control condition is satisfied.

In some instances, computing device 104 includes a set of quality-control conditions for more than one estimated characteristic. As a result, the genetic algorithm may be configured for a separate iteration for each estimated characteristic. If the set of quality-control conditions are not all satisfied, the computing device 104 may communicate with the sample distribution system(s) 111 and/or the associated user device in order to halt (e.g., or delay, in the event that the sample is altered to meet the quality-control conditions) distribution of the sample. If all of the set of quality-control conditions are satisfied, the computing device 104 may allow the distribution of the sample.

In some instances, the computing device 104 may further use an estimated characteristic in order to determine whether to allow, facilitate, inhibit or prevent a batch of samples from being distributed by the sample-distribution system 111. For example, in the event that at least an amount (e.g., a predefined threshold value or a majority) of samples within a batch of samples do not satisfy the quality-control condition, the batch of samples may be classified as an “unsatisfactory” batch. The computing device 104 may communicate with the sample distribution system 111 and/or the associated user device in order to halt distribution of any batches of samples that have been deemed to be “unsatisfactory”. In some instances, the “unsatisfactory” batches of samples are further altered to meet the quality-control conditions. In the event that at least a number (e.g., either a majority or a predefined threshold value) of samples within a batch of samples satisfies the quality-control condition, the batch of samples may be classified as a “satisfactory” batch. In such instances, the computing device 104 will only halt distribution of individual samples within a “satisfactory” batch that do not satisfy the quality-control condition. In other instances, the computing device 104 allows distribution of individual samples within a batch of samples that do not satisfy the quality-control condition as long as long as the batch of samples has been classified as “satisfactory”.

Furthermore, fulfillment or non-fulfillment of a quality-control condition may determine adjustment in the production process of future samples. If the quality-control condition is not satisfied, the sample production system may be altered such that components (e.g., an addition of a compound and/or percentage of a solute, removal of a compound and/or percentage of a solute, use of different configuration(s) for a sample production machine(s)) of the sample production system may be added, modified, or removed. For example, if a quality-control condition indicates the concentration of a solute within a sample is too high, the sample production system may adjust the addition of the solute for a lower concentration. In some instances, the sample production system may only be adjusted if a certain number (e.g. may be a predetermined threshold value) of samples are not satisfying a quality-control condition.

### III. Exemplary Method

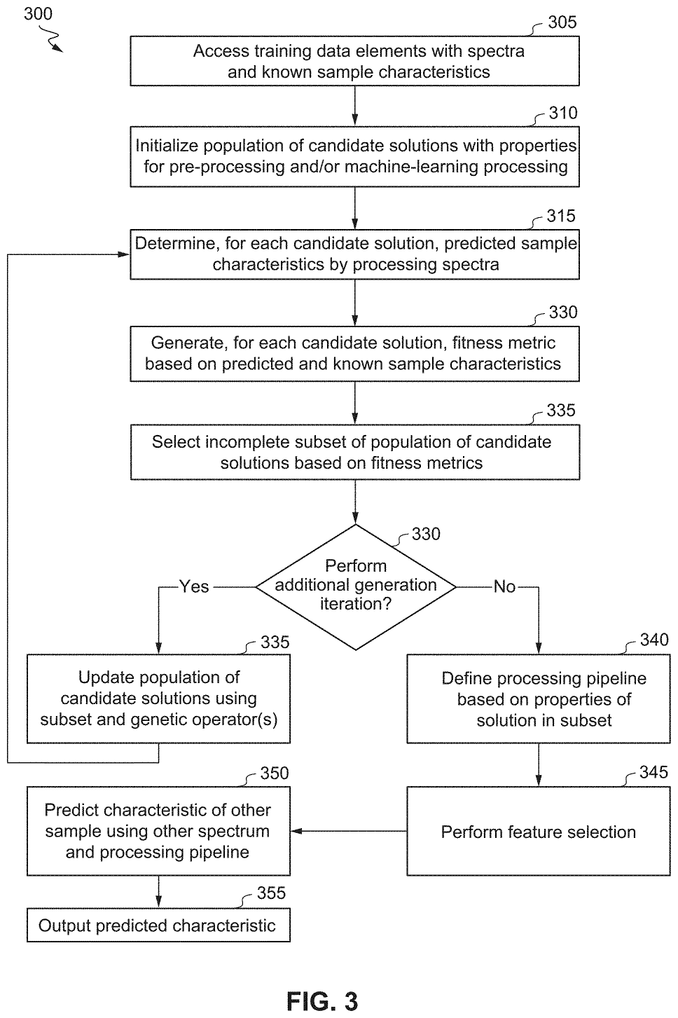

FIG. 3 shows an exemplary process 300 for using a genetic algorithm to facilitate quality-control processing of samples, in accordance with some embodiments of the invention. A computing device (e.g., such as computing device 104) executes process 300. At block 305, the computing device access a set of data. Each data element can include a spectrum and a known characteristic (e.g., a known physical or chemical characteristic) of a sample.

At block 310, the computing device initializes a population of candidate solutions. Each candidate solution can include a set of properties to specify a type, technique or variable for pre-processing a spectrum and/or processing the spectrum (or a pre-processed version thereof) using a machine-learning model.

At block 315, the computing device determines, for each candidate solution in the population and for each of at least some of the set of data elements, a predicted sample characteristic by transforming the spectrum of the data element in accordance with any pre-processing and machine-learning model as configured in accordance with the set of properties associated with the candidate solution. For example, a baseline and/or filter can be identified based on at least one of the set of properties and at least a portion of the data elements, and the baseline may be removed and/or a spectrum may be filtered using the baseline and/or filter. As another example, a type of machine-learning model may be selected and configured in accordance with at least some of the set of properties of the candidate solution, and the machine-learning model may further be configured using at least some of the data elements. Individual spectra can then be processed using the configured pre-processing and/or machine-learning model. In some instances, a first portion of the data set (e.g., a training subset) is used to determine or learn any data-dependent values, and the pre-processing and machine-learning model (configured with the data-dependent values and set of properties) are used to generate a predicted sample characteristic for each data element in one or more second portions of the data set (e.g., a validation subset and/or testing subset).

At block 320, the computing device generates a fitness metric for each candidate solute based on the predicted sample characteristics and the known sample characteristics. A fitness metric may include (for example) an error metric, a correlation metric and/or a pair-wise significance value. For example, a fitness metric may include a signal to noise ratio, a root-mean square error, R² value or p-value generated using a paired analysis. In some instances, the fitness metric is generated using a validation or testing subset of the data set. In some instances, the fitness metric is generated using a classification accuracy value of the predicted sample characteristic and the known sample characteristics (e.g., assigning a “satisfactory” label if a calculated error metric is in between a predetermined upper bound and a lower bound). In some instances, the fitness metric is configured such that low values and/or a “0” value represent that the candidate solution is better at predicting sample characteristics as compared to higher values. In some instances, the fitness metric is configured such that high values and/or a “1” value represent that the candidate solution is better at predicting sample characteristics as compared to lower values.

At block 325, the computing device selects an incomplete subset of the population of candidate solutions based on the fitness metrics. The incomplete subset may include a predefined number of candidate solutions (e.g., 1 or 3), a predefined percentage of the population of candidate solutions (e.g., 5% or 10%), or each candidate solution in the population that is associated with a fitness metric that is above (or below) a predefined threshold. The incomplete subset can be selected to include (for example) the candidate solution(s) that are associated with fitness metrics indicating better prediction performance relative to other candidate solutions not in the subset. For example, the subset can be selected to include two candidate solutions from the population that are associated with the lowest error-based fitness metrics in the population or that are associated with the highest correlation-based fitness metrics in the population.

At block 330, the computing device determines whether to perform an additional generation iteration. For example, it may be determined to perform an additional generation when a current generation count is less than a predefined number of generations to be assessed.

If the computing device determines that an additional generation iteration is to be performed, process 300 can proceed to block 335, where the population of candidate solutions can be updated using the subset and one or more genetic operators. Updating the population of candidate solutions can include replacing the population of candidate solutions with a new population of candidate solutions (e.g., each candidate solution in the new population being associated with a new set of properties). The new population can be generated by selecting, for each of the set of properties, a value (e.g., using a pseudo-random selection technique). The selection may be biased towards a value associated with the incomplete subset. The selection may use one or more genetic operators, such as a mutation operator, crossover operator and/or selection operator. Process 300 can then return to block 315 to evaluate the updated population of candidate solutions.

If the computing device determines, at block 330, that an additional generation iteration is not to be performed, process 300 can proceed to block 340, where a processing pipeline is defined based on a set of properties of a candidate solution in the subset. The processing pipeline can identify the type(s) of pre-processing to be performed (if any) and the type of machine-learning-model processing to be performed. In some instances, the processing pipeline includes particular variables, such as one or more unlearned variables defined by a property of the set of properties and/or one or more learned parameters defined based on the training data.

At block 345 the computing device performs, in the processing pipeline, a feature-selection process. The computing device identifies, from the input spectrum of a particular stage of the processing pipeline (e.g., such as prior predicting the characteristic of a sample), a set of wavenumbers and corresponding intensities from the input spectrum. The feature-selection process includes selecting from the set of wavenumbers, one or more wavenumbers and corresponding intensities (e.g., features) to be used in predicting the characteristic of the sample. By selecting wavenumbers, the computing device can reduce the quantity of intensities from the input spectrum that are used to predict the characteristic.

The feature-selection process includes generating a rank for each wavenumber of the set of wavenumbers. The rank may be generated using a regression analysis such as a partial least squares (PLS) regression. PLS outputs a set of components that describe a correlation between a wavenumber and other wavenumbers (e.g., indicative of a degree in which varying the intensity of a wavenumber varies the intensities of other wavenumbers). A rank is assigned to each wavenumber based on a relative ordering of the components of the partial least squares regression. The rank is indicative of a contribution of a wavenumber to the variability of the set of wavenumbers. A high ranking wavenumber indicates that varying the intensity of the wavenumber causes a corresponding variability in one or more other wavenumbers. A low ranking wavenumber indicates that varying the wavenumber will cause little or no change in the intensities of other wavenumbers. The wavenumbers of the spectrum are sorted according to the rank of each wavenumber. For instance, the wavenumbers are sorted from wavenumbers with a highest rank to wavenumbers with a lowest rank or vice versa.

The computing device defines a set of iterations with each iteration evaluating a different subset of the set of wavenumbers. The subset of wavenumbers of the first iteration includes all of the wavenumbers. The subset of wavenumbers of each subsequent iteration includes, the wavenumbers from the previous iteration minus a quantity of wavenumbers based on rank (e.g., lowest wavenumbers, highest wavenumbers, random sampling of wavenumbers, or the like). In one example, if the spectrum includes 1500 wavenumbers, then the subset of the first iterations includes 1500 wavenumbers, the subset of the second iteration includes the 1500 from the first iteration minus 25% of wavenumbers with a low rank (e.g., leaving 1125 wavenumbers remaining), the subset of the third iteration includes the 1125 from the first iteration minus the percentage of those wavenumbers having a low rank (e.g., leaving 825 wavenumbers remaining iteration), and so on.

The computing device evaluates each iteration of the set of iterations by defining a model-validation score for each iteration based on a cross-validation analysis as previously described in FIG. 2. Each score represents a degree to which processing spectra (in accordance with a processing pipeline) that include intensities for wavenumbers in the subset accurately predict a sample characteristic. The model-validation score of the first iteration (e.g., that includes the set of wavenumbers) is a baseline model-validation score that is compared to subsequent model-validation scores. Comparing model-validation scores to the baseline model-validation score provides an indicates the effect of removing wavenumbers on the accuracy of predicting a sample characteristic.

The feature-selection process then identifies a particular iteration from the predetermined quantity of iterations that has a model-validation score that is within a threshold deviation from the baseline model-validation score. For example, a threshold can be set to 0.020 (e.g., or any predetermined quantity based on the genetic algorithm, user input, a quantity of wavenumbers, the baseline model-validation score, combinations thereof, or the like). The computing device identifies a particular iteration having a model-validation score that is closest to the threshold from the baseline model-validation score. In some examples, the feature-selection process identifies a particular iteration having a model-validation score that is closest to the threshold from the baseline model-validation score without exceeding the threshold.

In some instances, the computing device compares the model-validation score derived for each iteration to the baseline model-validation score before moving on to the next iteration. Upon detecting an iteration having a model-validation score that is greater than the threshold deviation, the feature-selection process identifies the previous iteration (e.g., the iteration before the iteration having a model-validation score that is greater than the threshold deviation from the baseline model-validation score) as the particular iteration. In those instances, the feature-selection process is configured to perform a predetermined quantity of iterations, but terminate early upon identifying the particular iteration to reduce the number of analyzed iterations.

The intensities that correspond to the wavenumbers of the particular iteration can be used to predict the characteristic the sample. Since fewer wavenumbers are used, the overall complexity of the predictor (e.g., machine-learning model, or the like as previously described) can be reduced without impacting the performance of the predictor (e.g., prediction accuracy, etc.).

When processing subsequent spectra, the computing device selects the intensities of the new spectra at the same wavenumbers identified by the feature-selection process for use in predicting the characteristic. Wavenumbers and corresponding intensities that do not correspond to the wavenumbers identified by the feature-selection process may be omitted from further processing by the processing pipeline. Alternatively, wavenumbers and corresponding intensities that do not correspond to the wavenumbers identified by the identified by the feature-selection process may be removed from the new spectrum. The feature-selection process described in block 340 may be performed once to select the wavenumbers that can be used to predict the characteristic in subsequent spectra.

In some instances, the computing device executes the feature-selection process for each new spectrum for which a characteristic is to be predicted. In those instances, each execution of the processing pipeline for a new spectra includes a feature-selection process that occurs prior to predicting the characteristic.

The feature-selection process can be performed as a stage of the processing pipeline prior to generation of the prediction of the characteristic (e.g., as described in block 345). Alternatively, the feature-selection process can be performed within the genetic algorithm (e.g., as gene that persists across generations). In those instances, the feature-selection process is defined within a candidate solution of the population of candidate solutions. The feature-selection process can be varied by the genetic algorithm by, for example, varying the predetermined quantity if iterations to be performed by the feature-selection process, varying the predetermined quantity of wavenumbers to be removed during each iteration, varying the percentage of waveforms to be removed during each iterations, varying the threshold from the baseline model-validation score to identify the particular iteration, combinations thereof, or the like, in candidate solutions and/or across generations.

For example, the feature-selection process including a predetermined set of attributes (e.g., that correspond to quantity of iterations, percentage of wavenumbers to be removed during each iteration, etc.) are included within one or more candidate solutions. In some instances, the feature-selection process in some candidate solutions may be different from the feature-selection process in other candidate solutions. For instance, a feature-selection process included in one or more candidate solutions may include 12 iterations, and a feature-selection process included in one or more candidate solutions may include 9 iterations. The genetic algorithm identifies whether the feature-selection process is to be included in a candidate solution and if so, the set of attributes that correspond to an improved prediction of the characteristic (e.g., more accurate, etc.).

The computing device, at block 350, uses the processing pipeline to process another spectrum associated with another sample to predict a characteristic of the other sample. The other sample may correspond to one not represented in the data set used to evaluate various candidate solutions. After the new spectrum is processed by the processing pipeline but before the prediction of the characteristic is made, the wavenumbers are selected for use in predicting the characteristic. The wavenumbers selected correspond to the wavenumbers identified by the feature-selection process of block 340. Non-selected wavenumbers are omitted from further processing or otherwise not used in predicting the characteristic.

At block 355, the computing device outputs the predicted characteristic. For example, the predicted characteristic is presented locally or transmitted to another device. An identifier of the other sample may further be output in association with the predicted characteristic.

### IV. Examples

### A. Example 1—Candidate Solution Population for a Single Generation

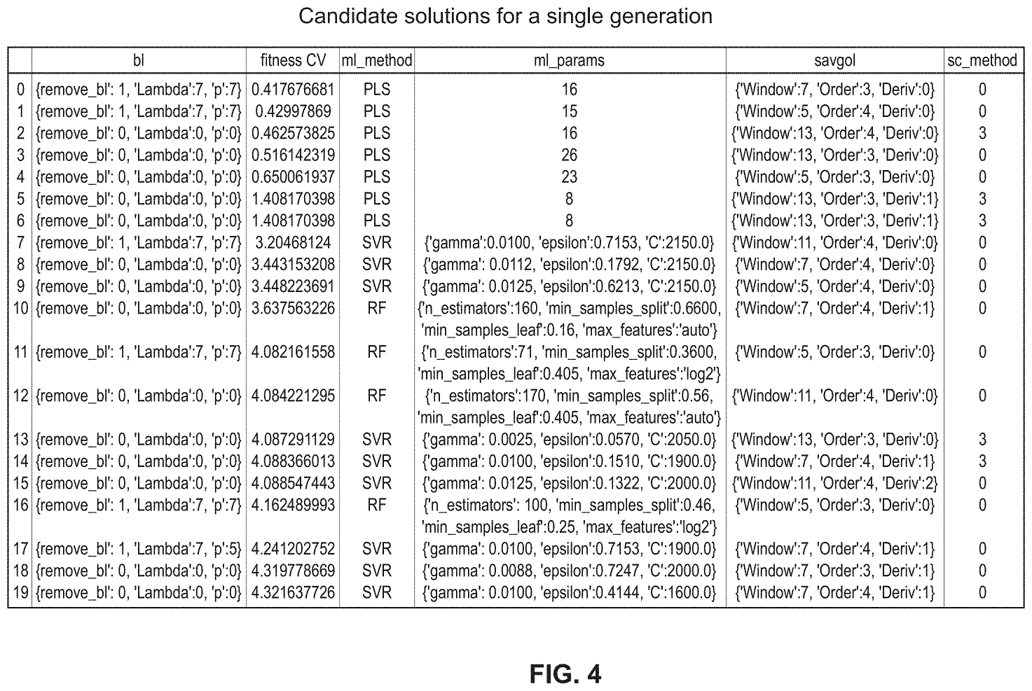

FIG. 4 shows an exemplary population of 20 candidate solutions generated for a single generation. Each candidate solution includes a value for each of the following properties:
Whether Asymmetric Least Squares baseline removal is performed, including the following parameters:
   A λ value for Asymmetric Least Squares baseline removal;
   A p value for Asymmetric Least Squares baseline removal;
A type of machine-learning model to be used in processing: partial least squares (e.g., principal component analysis, PLS discriminant analysis, etc.), random forest (e.g., boosted tree models, such as AdaBoost or XGBoost; splitting random forest; etc.) or support vector machine (e.g., C-SVM classification, nu-SVM classification, epsilon-SVM regression, etc.);
Hyperparameters for the machine-learning model, including:
   If the model type is a partial least squares model: a number of machine-learning parameters (i.e. a number of principal components to calculate);
   If the model type is a random-forest model: a minimum number of samples required to be a leaf node;
   If the model type is a random-forest model: a minimum number of samples required to split an internal node;
   If the model type is a support vector machine model: regularization and kernel parameter values;
Whether a Savitzky-Golay (“savgol”) smoothing is performed;
A window size for smoothing pre-processing;
A polynomial order for smoothing pre-processing;
A derivative order for smoothing pre-processing; and
A selection of preprocessing techniques including but not limited to mean centering and diverse scaling strategies such as the Standard Normal Variate method; performing scaling using a maximum intensity value; performing scaling using L1 metric; or not performing scaling.

In addition, each candidate solution has been given a fitness metric value (e.g., depicted as the “fitness CV” column) based upon how accurately each candidate solution can estimate a characteristic. The best performing candidate solutions (e.g., with the lowest fitness metric values) are ranked in descending order with candidate solution 0 as the most accurate and candidate solution 19 as the least accurate. A genetic algorithm may choose any of the top candidate solutions (e.g., such as candidate solution 0 and/or candidate solution 1) to be included within a new population of candidate solutions for a next generation.

### B. Example 2—Lactate-Concentration Labels

A training data set was defined to include 5000 Raman spectra (each collected using and corresponding to an individual sample) and 5000 labels. Each label can identify a sample characteristic, which, in this example, that identify an amount of lactate within the corresponding sample. Each sample being monitored included eukaryotic cell culture. An initial set of candidate solutions was defined to have 10 candidate solutions, each being associated with a value for each of the same properties from the candidate solutions in Example 1.

A genetic algorithm was then used to evaluate each of the 10 candidate solutions. The training data set was used to learn particular parameters (e.g., to identify a particular baseline to be removed using the Asymmetric Least Squares technique when a candidate solution set of properties indicate that baseline removal is to be performed). For each candidate solution, a candidate processing pipeline was defined in accordance with the candidate solution's set of properties and any learned parameters. The fitness metric was calculated by generating, for each of 500 Raman spectra in a validation data set, a predicted label using the candidate solution's candidate processing pipeline and comparing the predicted label to a known label.

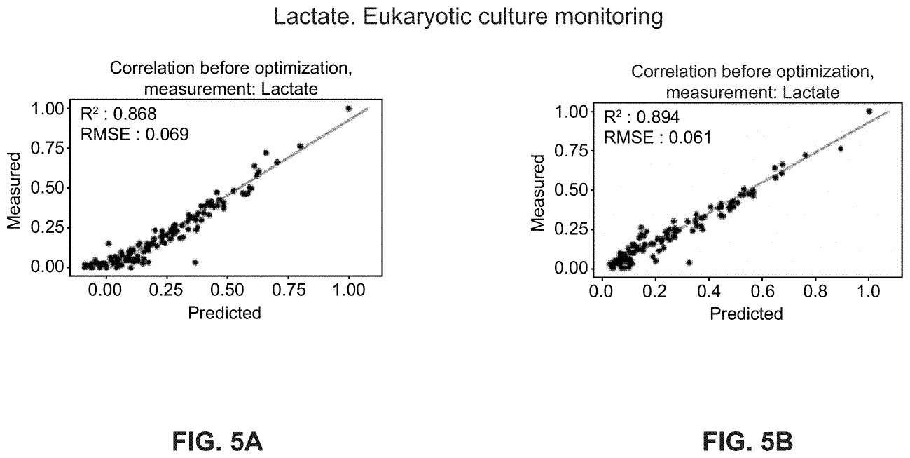

FIG. 5A shows comparisons between the measured label values of the lactate concentration and the predicted label values of the lactate concentration generated by the exemplary candidate solution's candidate processing pipeline. For this candidate processing pipeline, the R² value was determined to be 0.868, and the root-mean square error was calculated to be 0.069 for a test data set.

FIG. 5A pertains to an exemplary candidate solution from a first generation that includes the following configurations:
Baseline removal: None
Savitzky-Golay smoothing is to be performed using a window size of 15, a polynomial order of 2, and a derivative order of 1.
Scaling is to be performed in accordance with the Standard Normal Variate row-wise method.
The machine-learning model to be used is partial least squares regression with 6 components.

A subset of the generation's candidate solutions was defined to include the 2 candidate solutions, from amongst the 10 candidate solutions, associated with the highest fitness metrics. Properties from the candidate solutions in the subset were input into a mutation algorithm, and a set of properties for each of 10 new candidate solutions for a second generation were then defined. The candidate solutions were assessed and new generations were defined in a similar manner until fitness metrics were generated for each of 30 generations were generated. A single candidate solution was then selected from amongst the candidate solutions of the 30^{th} generation by identifying the candidate solution associated with the highest fitness metric for the generation.

FIG. 5B shows comparisons between the measured label values of the lactate concentration and the predicted label values of the lactate concentration generated by a single candidate solution after the 30th generation. The exemplary candidate solution has the following configurations:
Asymmetric Least Squares baseline removal is to be performed with 2=4 and p=7.
Savitzky-Golay smoothing is to be performed using a window size of 9, a polynomial order of 2, and a derivative order of 0.
Scaling is to be performed in accordance with the Standard Normal Variate method.
The machine-learning model to be used is a random forest where a minimum number of samples to be a leaf node was 7, a maximum number of features was 300, and a minimum number of samples to split an internal node was 5. The random forest includes 100 estimators.

For this processing pipeline, the R² value was determined to be 0.894, and the root-mean square error calculated for a test data set was 0.061. Thus, the agreement between the predicted and actual labels was higher for the selected single candidate solution (identified after 30 generations) as compared to the label agreement from the first generation's exemplary candidate solution. Further, the error of the predicted labels was lower for the selected single candidate solution (identified after 30 generations) as compared to the error of the first generation's exemplary candidate solution.

### C. Example 3—Glucose-Concentration Labels

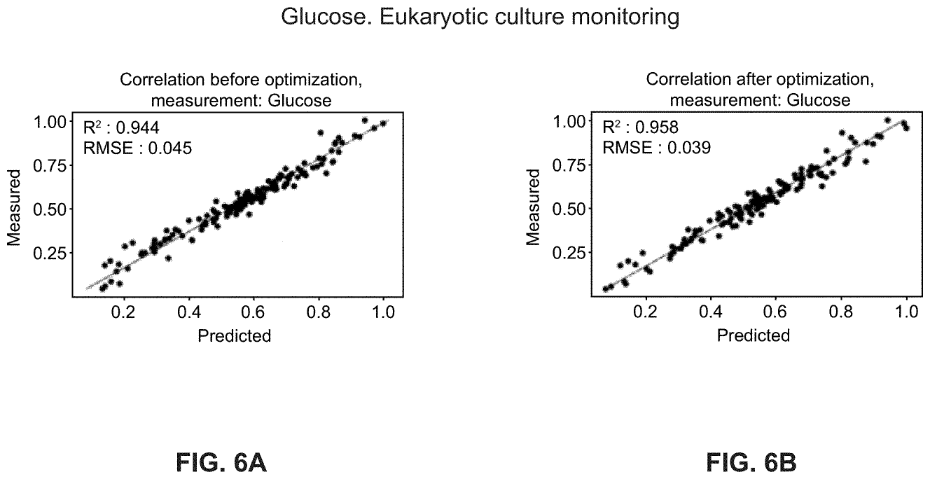

FIGS. 6A and 6B show exemplary comparisons between the measured label values of pH and the predicted label values of glucose-concentration for an exemplary candidate solution from a first generation and an exemplary candidate solution from a 30 generation. A similar processing was performed in this example as was performed in Example 2. The labels identify an amount of glucose in the samples rather than an amount of lactate in the samples, and an eukaryotic cell culture was being monitored. Each of FIGS. 6A and 6B show comparisons between actual and estimated labels. FIG. 6A pertains to an exemplary candidate solution from a first generation, and FIG. 6B pertains to the single candidate solution (identified after 30 generations).

The candidate processing pipeline for the exemplary candidate solution in the first generation included the following configurations:
No baseline removal is to be performed.
Savitzky-Golay smoothing on a first derivative is to be performed using a window size of 15, a polynomial order of 2, and a derivative order of 1.
Scaling is to be performed in accordance with the Standard Normal Variate method.
The machine-learning model to be used is partial least squares with 8 principal components.

The candidate processing pipeline for the single candidate solution selected after the 30th generation included the following configurations:
Asymmetric Least Squares baseline removal is to be performed with 2=4 and p=7.
Savitzky-Golay smoothing on a first derivative is to be performed using a window size of 13, a polynomial order of 2, and a derivative order of 1.
Scaling is not to be performed.
The machine-learning model to be used is partial least squares with 9 principal components.

The R² value was higher for the single candidate solution selected after the 30^{th} generation as compared to that of the exemplary first-generation candidate solution (R²=0.958 versus R²=0.944 respectively). Further, the test-set errors for the single candidate solution selected after the 30^{th} generation were lower as compared to those of the exemplary first-generation candidate solution (RMSE=0.039 versus RMSE=0.045 respectively).

Notably, some of the properties of the selected single candidate solution pertaining to this Example differed from corresponding properties of the selected single candidate solution pertaining to Example 2. For example, the machine-learning model selected in this example was a partial least squares model, while the machine-learning model selected for Example 2 was a random-forest model. This may indicate that various pre-processing and processing techniques and/or configurations are differentially effective for predicting a label depending on the type of label being predicted.

### D. Example 4—pH Labels

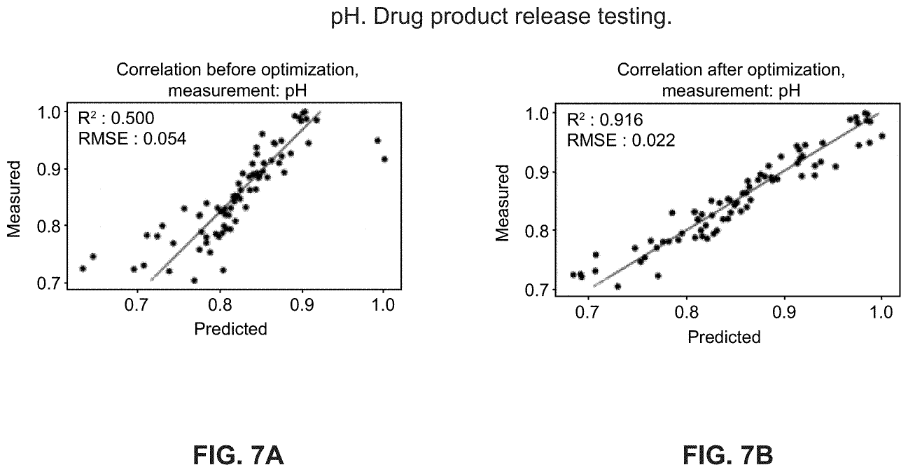

FIGS. 7A and 7B show exemplary comparisons between the measured label values of pH and the predicted label values of pH for an exemplary candidate solution from a first generation and an exemplary candidate solution from a 30th generation. A similar processing was performed in this example as was performed in Example 2. The labels of Example 4 identify a pH of the samples (e.g., in this context, biopharmaceutical material in a formulation buffer) rather than an amount of lactate in eukaryotic cell culture samples. In this example, the measurement is a quality attribute that can determine a release and distribution of a sample to subjects. Each of FIGS. 7A and 7B show comparisons between actual and estimated labels.

FIG. 7A pertains to an exemplary candidate solution from a first generation that included the following configurations:
No baseline removal is to be performed.
Savitzky-Golay smoothing on a first derivative is to be performed using a window size of 15, a polynomial order of 2, and a derivative order of 1.
Scaling is to be performed in accordance with the Standard Normal Variate row-wise method.
The machine-learning model to be used is partial least squares with 6 principal components.

FIG. 7B pertains to the single candidate solution (identified after 30 generations) included the following configurations:
Asymmetric Least Squares baseline removal is to be performed with 2=6 and p=3.
Savitzky-Golay smoothing on a first derivative is to be performed using a window size of 5, a polynomial order of 3, and a derivative order of 0.
Scaling is not to be performed.
The machine-learning model to be used is partial least squares with 20 principal components.

The R² value was higher for the single candidate solution selected after the 30^{th} generation as compared to that of the exemplary first-generation candidate solution (R²=0.916 versus R²=0.500 respectively). Further, the test-set errors for the single candidate solution selected after the 30^{th} generation were lower as compared to those of the exemplary first-generation candidate solution (RMSE=0.022 versus RMSE=0.054 respectively).

### E. Example 5—Osmolality Labels

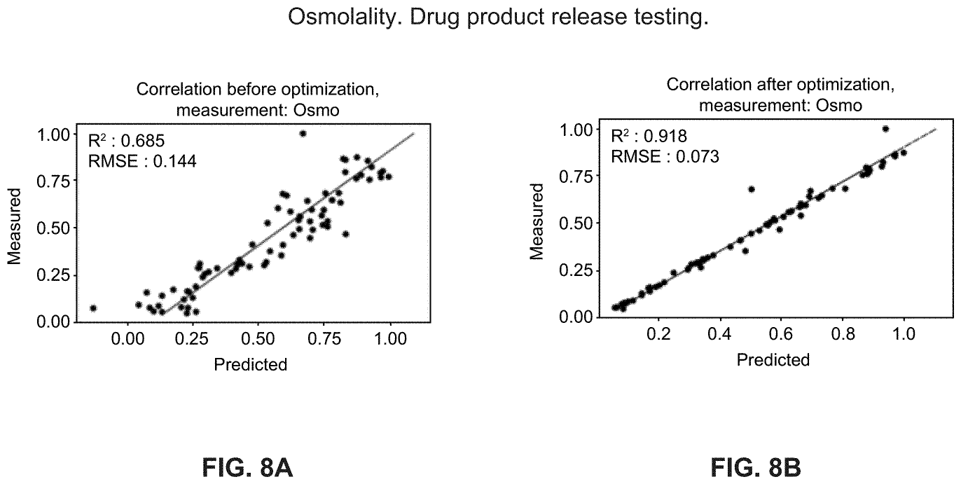

FIGS. 8A and 8B show exemplary comparisons between the measured label values of osmolality and the predicted label values of osmolality for an exemplary candidate solution from a first generation and an exemplary candidate solution from a 30^{th} generation. A similar processing was performed in this example as was performed in Example 2. The labels of Example 5 labels identify an osmolality of the samples (e.g., in this context, solute concentration of biopharmaceutical material in a formulation buffer). Each of FIGS. 8A and 8B show comparisons between actual and estimated labels.

FIG. 8A pertains to an exemplary candidate solution from a first generation that included the following configurations:
No baseline removal is to be performed.
Savitzky-Golay smoothing on a first derivative is to be performed with a window size of 15, polynomial order of 2, derivative order of 1.
Scaling is to be performed in accordance with the Standard Normal Variate row-wise method.
The machine-learning model to be used is partial least squares with 8 principal components.

FIG. 8B pertains to the single candidate solution (identified after 30 generations) included the following configurations:
Asymmetric Least Squares baseline removal is to be performed with 2=4 and p=7.
Savitzky-Golay smoothing on a first derivative is to be performed with a window size of 5, polynomial order of 3, derivative order of 0.
Scaling is to be performed in accordance with the Standard Normal Variate row-wise method.
The machine-learning model to be used is support vector machine where C: 2100, γ: 0.01584.

The R² value was higher for the single candidate solution selected after the 30^{th} generation as compared to that of the exemplary first-generation candidate solution (R²=0.918 versus R²=0.685 respectively). Further, the test-set errors for the single candidate solution selected after the 30^{th} generation were lower as compared to those of the exemplary first-generation candidate solution (RMSE=0.073 versus RMSE=0.144 respectively).

### F. Example 6—Antibody Oxidation Labels

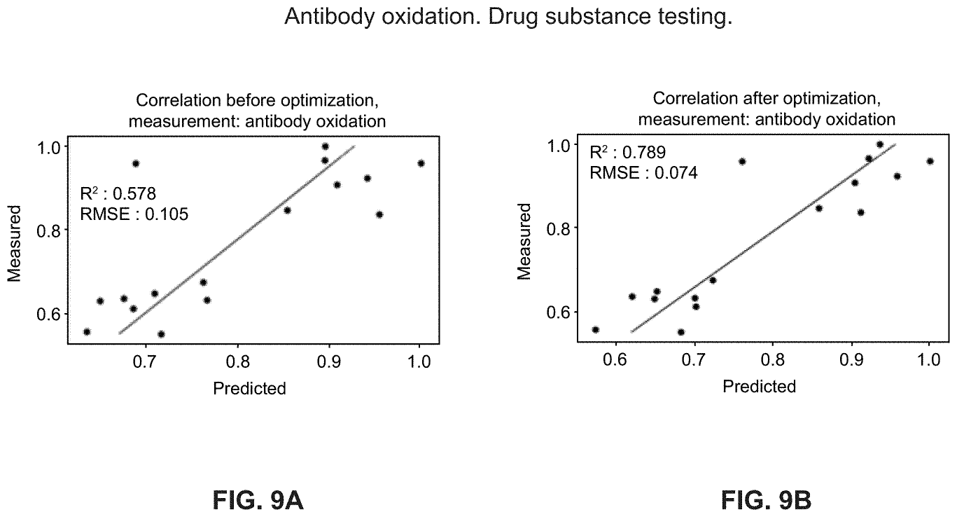

FIGS. 9A and 9B show exemplary comparisons between the measured label values of antibody oxidation and the predicted label values of antibody oxidation for an exemplary candidate solution from a first generation and an exemplary candidate solution from a 30^{th} generation. A similar processing was performed in this example as was performed in Example 2. The labels of Example 6 identify an estimated antibody oxidation of the samples (e.g., in this context, an estimation of therapeutic antibody functionality). Each of FIGS. 9A and 9B show comparisons between actual and estimated labels.

FIG. 9A pertains to an exemplary candidate solution from a first generation that included the following configurations:
No baseline removal is to be performed.
Savitzky-Golay smoothing on a first derivative is to be performed with a window size of 15, polynomial order of 2, derivative order of 1.
Scaling is to be performed in accordance with the Standard Normal Variate row-wise method.
The machine-learning model to be used is partial least squares with 5 principal components.

FIG. 9B pertains to the single candidate solution (identified after 30 generations) included the following configurations:
No baseline removal is to be performed.
Savitzky-Golay smoothing on a first derivative is to be performed with a window size of 5, polynomial order of 4, derivative order of 0.
Scaling is to be performed in accordance with the Standard Normal Variate row-wise method.
The machine-learning model to be used is partial least squares regression with 10 principle components.

The R² value was higher for the single candidate solution selected after the 30^{th} generation as compared to that of the exemplary first-generation candidate solution (R²=0.789 versus R²=0.578 respectively). Further, the test-set errors for the single candidate solution selected after the 30^{th} generation were lower as compared to those of the exemplary first-generation candidate solution (RMSE=0.074 versus RMSE=0.105 respectively).

### G. Example 7—Glycan G0F-N Labels

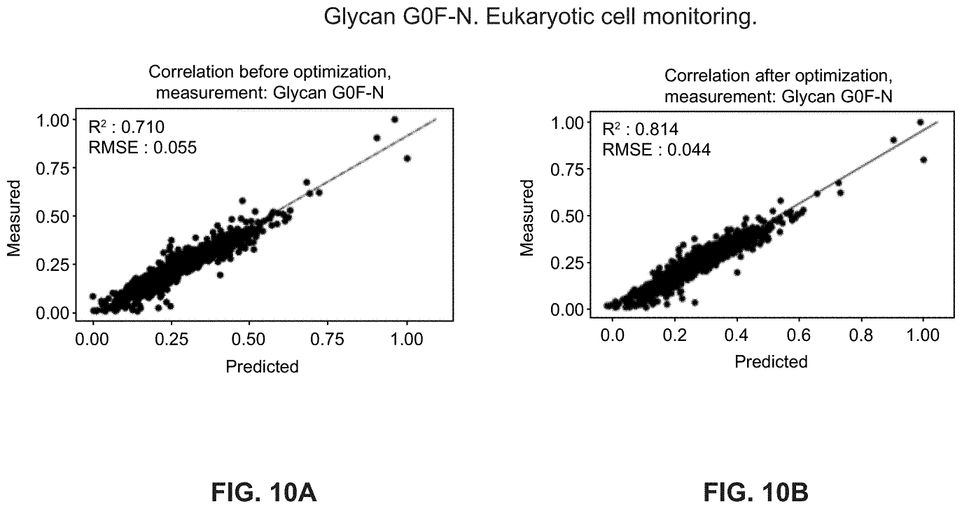

FIGS. 10A and 10B show exemplary comparisons between the measured label values of glycan G0F-N and the predicted label values of glycan G0F-N for an exemplary candidate solution from a first generation and an exemplary candidate solution from a 30^{th} generation. A similar processing was performed in this example as was performed in Example 2. The labels of Example 7 identify an estimated glycan GOF-N of the samples. Each of FIGS. 10A and 10B show comparisons between actual and estimated labels.

FIG. 10A pertains to an exemplary candidate solution from a first generation that included the following configurations:
No baseline removal is to be performed.
Savitzky-Golay smoothing on a first derivative is to be performed with a window size of 15, polynomial order of 2, derivative order of 1.
Scaling is to be performed in accordance with the Standard Normal Variate row-wise method.
The machine-learning model to be used is partial least squares with 5 principal components.

FIG. 10B pertains to the single candidate solution (identified after 30 generations) included the following configurations:
Asymmetric Least Squares baseline removal is to be performed, with 2=6 and p=9.
Savitzky-Golay smoothing on a first derivative is to be performed with a window size of 5, polynomial order of 3, derivative order of 0.
Scaling is to be performed in accordance with the Standard Normal Variate row-wise method.
The machine-learning model to be used is support vector machine where C: 2400, γ: 0.0006.

The R² value was higher for the single candidate solution selected after the 30^{th} generation as compared to that of the exemplary first-generation candidate solution (R²=0.814 versus R²=0.710 respectively). Further, the test-set errors for the single candidate solution selected after the 30^{th} generation were lower as compared to those of the exemplary first-generation candidate solution (RMSE=0.044 versus RMSE=0.055 respectively).

### H. Example 8—HMWF Labels

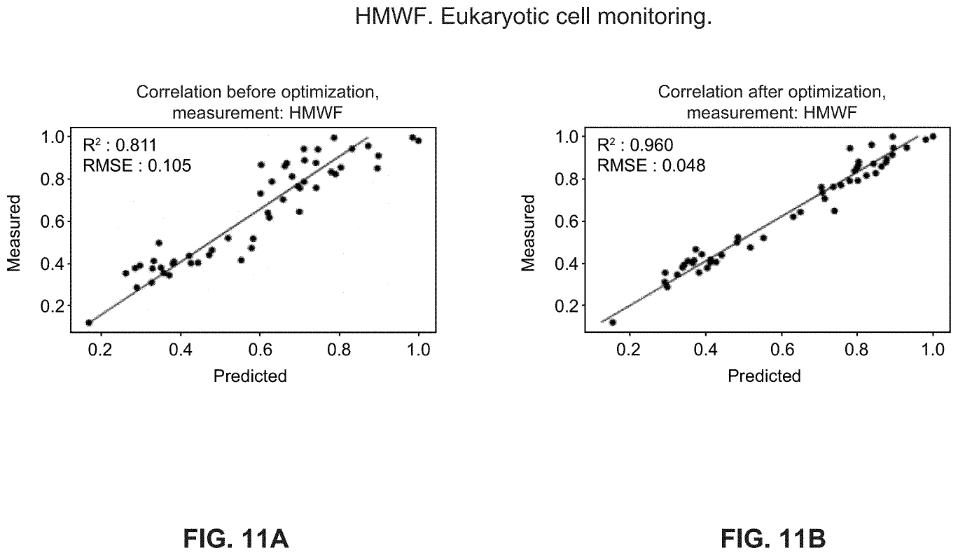

FIGS. 11A and 11B show exemplary comparisons between the measured label values of high-molecular-weight forms (HMWF) and the predicted label values of HMWF for an exemplary candidate solution from a first generation and an exemplary candidate solution from a 30^{th} generation. A similar processing was performed in this example as was performed in Example 2. The labels of Example 8 identify an estimated HMWF of the samples. Each of FIGS. 11A and 11B show comparisons between actual and estimated labels.

FIG. 11A pertains to an exemplary candidate solution from a first generation that included the following configurations:
No baseline removal is to be performed.
Savitzky-Golay smoothing on a first derivative is to be performed with a window size of 15, polynomial order of 2, derivative order of 1.
Scaling is to be performed in accordance with the Standard Normal Variate row-wise method.
The machine-learning model to be used is partial least squares with 8 principal components.

FIG. 11B pertains to the single candidate solution (identified after 30 generations) included the following configurations:
Asymmetric Least Squares baseline removal is to be performed with 2=7 and p=3.
Savitzky-Golay smoothing on a first derivative is to be performed with a window size of 11, polynomial order of 3, derivative order of 0.
Scaling is to be performed in accordance with the Standard Normal Variate row-wise method.
The machine-learning model to be used is support vector machine where C: 2100, γ: 0.1.

The R² value was higher for the single candidate solution selected after the 30^{th} generation as compared to that of the exemplary first-generation candidate solution (R²=0.960 versus R²=0.811 respectively). Further, the test-set errors for the single candidate solution selected after the 30^{th} generation were lower as compared to those of the exemplary first-generation candidate solution (RMSE=0.048 versus RMSE=0.105 respectively).

### I. Example 9—Bispecific Assembly Labels

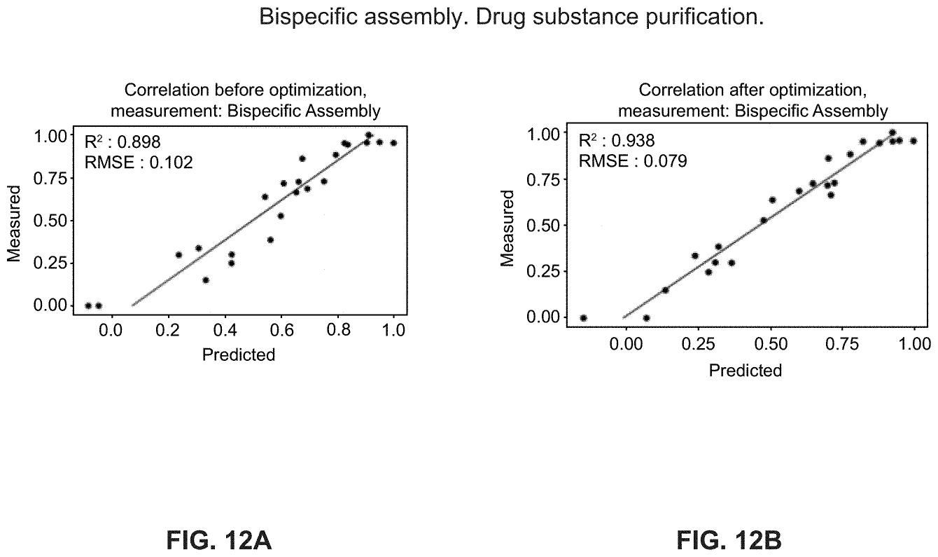

FIGS. 12A and 12B show exemplary comparisons between the measured label values of bispecific assembly and the predicted label values of bispecific assembly for an exemplary candidate solution from a first generation and an exemplary candidate solution from a 30^{th} generation. A similar processing was performed in this example as was performed in Example 2. The labels of Example 9 identify an estimation of bispecific assembly of antibodies in the samples (e.g., the percent of assembled bispecific antibody as a decimal fraction measured by reverse-phase mass spectrometry). Each of FIGS. 12A and 12B show comparisons between actual and estimated labels.

FIG. 12A pertains to an exemplary candidate solution from a first generation that included the following configurations:
No baseline removal is to be performed.
Savitzky-Golay smoothing on a first derivative is to be performed with a window size of 15, polynomial order of 2, derivative order of 1.
Scaling is to be performed in accordance with the Standard Normal Variate row-wise method.
The machine-learning model to be used is partial least squares with 6 principal components.

FIG. 12B pertains to the single candidate solution (identified after 30 generations) included the following configurations:
No baseline removal is to be performed.
Savitzky-Golay smoothing on a first derivative is to be performed with a window size of 13, polynomial order of 2, derivative order of 0.
Scaling is to be performed in accordance with the Standard Normal Variate row-wise method.
The machine-learning model to be used is partial least squares with 10 principal components.

The R² value was higher for the single candidate solution selected after the 30^{th} generation as compared to that of the exemplary first-generation candidate solution (R²=0.938 versus R²=0.898 respectively). Further, the test-set errors for the single candidate solution selected after the 30^{th} generation were lower as compared to those of the exemplary first-generation candidate solution (RMSE=0.079 versus RMSE=0.102 respectively).

### J. Example 10—Abundance of Viable Cells Assembly Labels

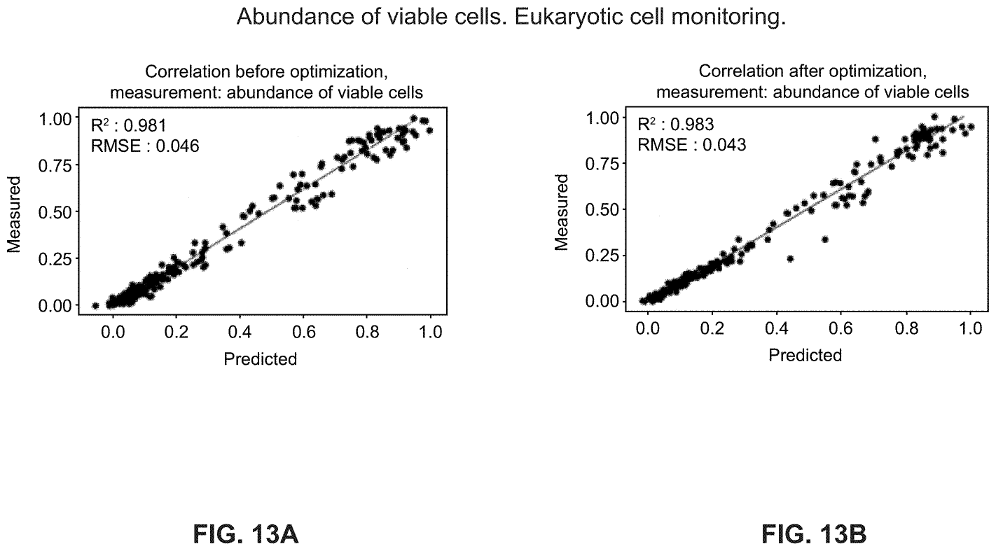

FIGS. 13A and 13B show exemplary comparisons between the measured label values of cell viability and the predicted label values of cell viability for an exemplary candidate solution from a first generation and an exemplary candidate solution from a 30^{th} generation. A similar processing was performed in this example as was performed in Example 2. The labels of Example 10 identify an estimation of an abundance of viable cells in the sample. Each of FIGS. 13A and 13B show comparisons between actual and estimated labels.

FIG. 13A pertains to an exemplary candidate solution from a first generation that included the following configurations:
No baseline removal is to be performed.
Savitzky-Golay smoothing on a first derivative is to be performed with a window size of 15, polynomial order of 2, derivative order of 1.
Scaling is to be performed in accordance with the Standard Normal Variate row-wise method.
The machine-learning model to be used is partial least squares with 11 principal components.

FIG. 13B pertains to the single candidate solution (identified after 30 generations) included the following configurations:
No baseline removal is to be performed.
Savitzky-Golay smoothing on a first derivative is to be performed with a window size of 15, polynomial order of 2, derivative order of 1.
Scaling is to be performed in accordance with the Standard Normal Variate row-wise method.
The machine-learning model to be used is support vector machine where C: 1550, γ: 0.0016.

The R² value was higher for the single candidate solution selected after the 30^{th} generation as compared to that of the exemplary first-generation candidate solution (R²=0.981 versus R²=0.983 respectively). Further, the test-set errors for the single candidate solution selected after the 30^{th} generation were lower as compared to those of the exemplary first-generation candidate solution (RMSE=0.043 versus RMSE=0.046 respectively).

### K. Example 11—Abundance of Dead Cells Assembly Labels

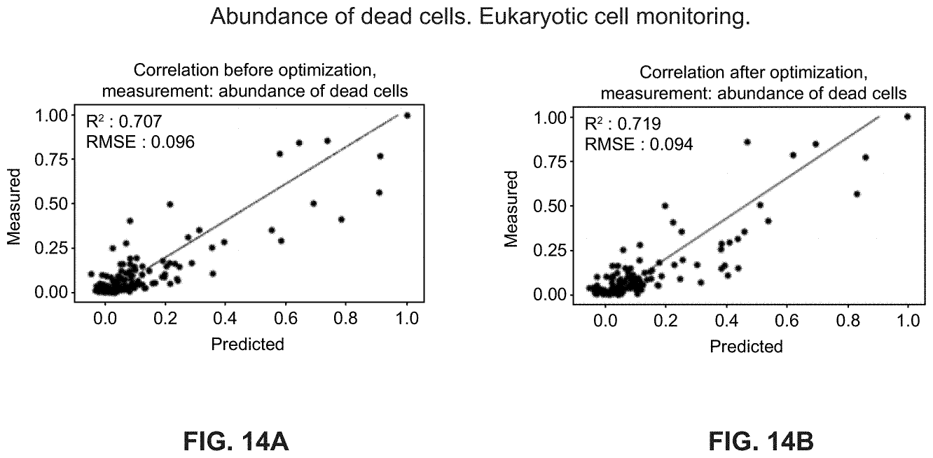

FIGS. 14A and 14B show exemplary comparisons between the measured label values of a quantity of dead cells and the predicted label values of a residual moisture content for an exemplary candidate solution from a first generation and an exemplary candidate solution from a 30^{th} generation. A similar processing was performed in this example as was performed in Example 2. The labels of Example 11 identify an estimation of an abundance of dead cells in the sample. Each of FIGS. 14A and 14B show comparisons between actual and estimated labels.

FIG. 14A pertains to an exemplary candidate solution from a first generation that included the following configurations:
No baseline removal is to be performed.
Savitzky-Golay smoothing on a first derivative is to be performed with a window size of 15, polynomial order of 2, derivative order of 1.
Scaling is to be performed in accordance with the Standard Normal Variate row-wise method.
The machine-learning model to be used is partial least squares with 12 principal components.

FIG. 14B pertains to the single candidate solution (identified after 30 generations) included the following configurations:
No baseline removal is to be performed.
Savitzky-Golay smoothing on a first derivative is to be performed, window size of 13, polynomial order of 2, derivative order of 1.
Scaling is to be performed in accordance with the Standard Normal Variate row-wise method.
The machine-learning model to be used is partial least squares with 8 principal components.

The R² value was higher for the single candidate solution selected after the 30^{th} generation as compared to that of the exemplary first-generation candidate solution (R²=0.719 versus R²=0.707 respectively). Further, the test-set errors for the single candidate solution selected after the 30^{th} generation were lower as compared to those of the exemplary first-generation candidate solution (RMSE=0.094 versus RMSE=0.096 respectively).

### L. Example 12—Residual Moisture Content Labels

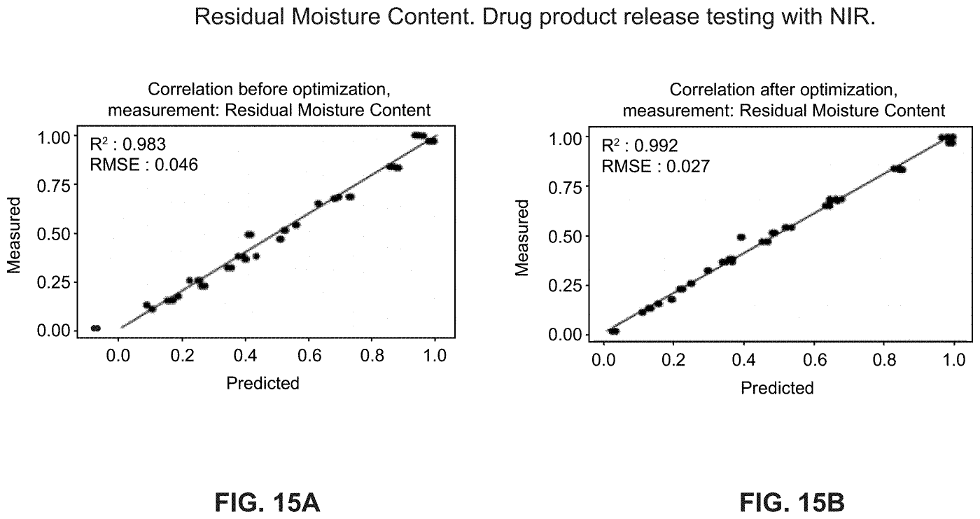

FIGS. 15A and 15B show exemplary comparisons between the measured label values of a residual moisture content and the predicted label values of a residual moisture content residual moisture content for an exemplary candidate solution from a first generation and an exemplary candidate solution from a 30^{th} generation. A similar processing was performed in this example as was performed in Example 2. The labels of Example 12 identify an estimation of residual moisture content of the sample. Each of FIGS. 15A and 15B show comparisons between actual and estimated labels.

FIG. 15A pertains to an exemplary candidate solution from a first generation that included the following configurations:
No baseline removal is to be performed.
Savitzky-Golay smoothing on a first derivative is to be performed with a window size of 11, polynomial order of 4, derivative order of 0.
Scaling is to be performed in accordance with the Standard Normal Variate row-wise method.
The machine-learning model to be used is partial least squares with 2 principal components.

FIG. 15B pertains to the single candidate solution (identified after 30 generations) included the following configurations:
Asymmetric Least Squares baseline removal is to be performed with 2=5 and p=9.
Savitzky-Golay smoothing on a first derivative is to be performed with a window size of 11, polynomial order of 4, derivative order of 1.
Scaling is to be performed in accordance with the Standard Normal Variate row-wise method.
The machine-learning model to be used is support vector machine where C: 2400, γ: 0.005, ε=0.066.

The R² value was higher for the single candidate solution selected after the 30^{th} generation as compared to that of the exemplary first-generation candidate solution (R²=0.992 versus R²=0.983 respectively). Further, the test-set errors for the single candidate solution selected after the 30^{th} generation were lower as compared to those of the exemplary first-generation candidate solution (RMSE=0.027 versus RMSE=0.039 respectively).

### M. Example 13—Manipulating Raw Spectra Characteristics with Preprocessing

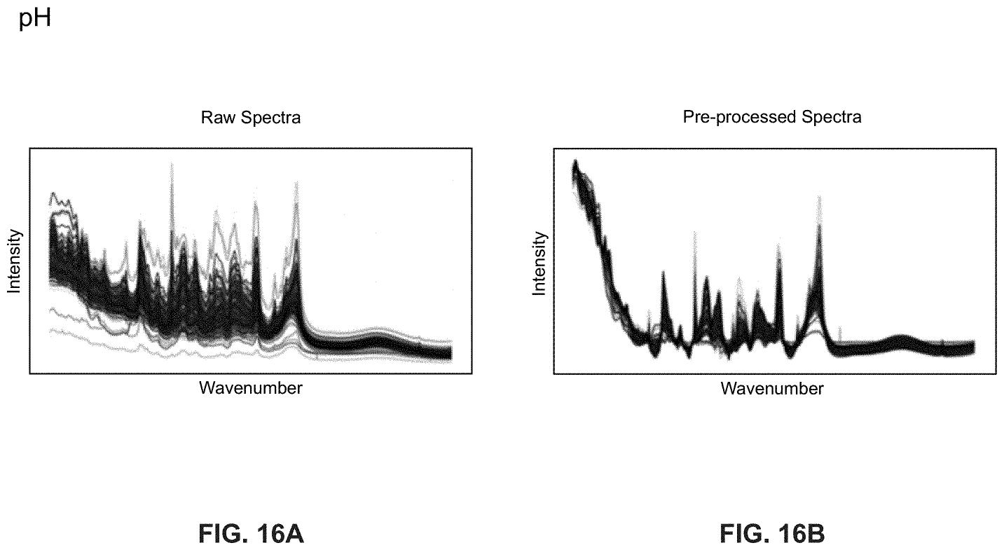

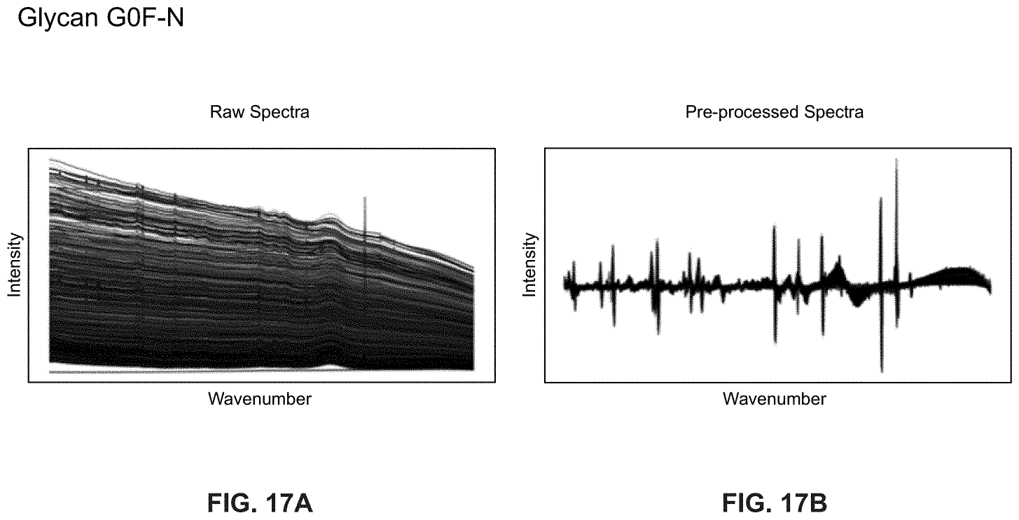

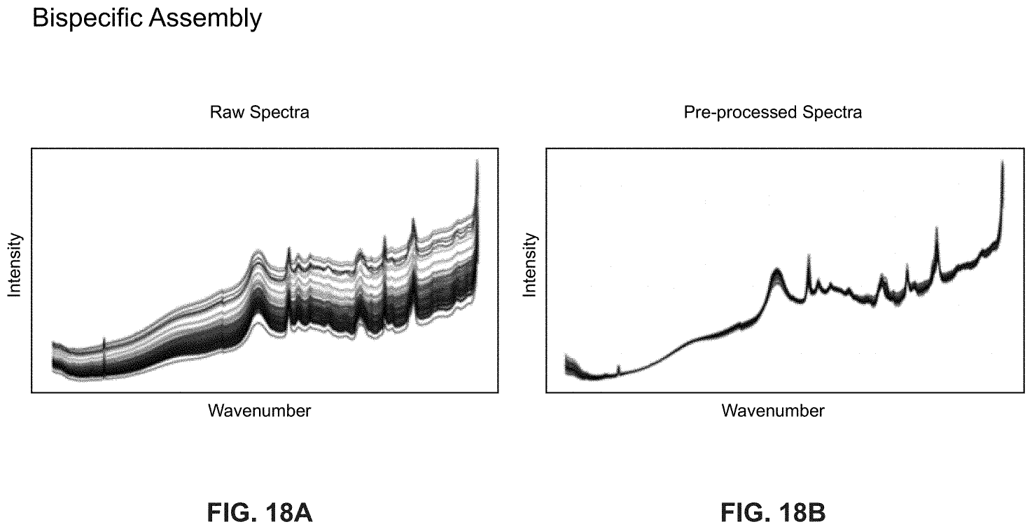

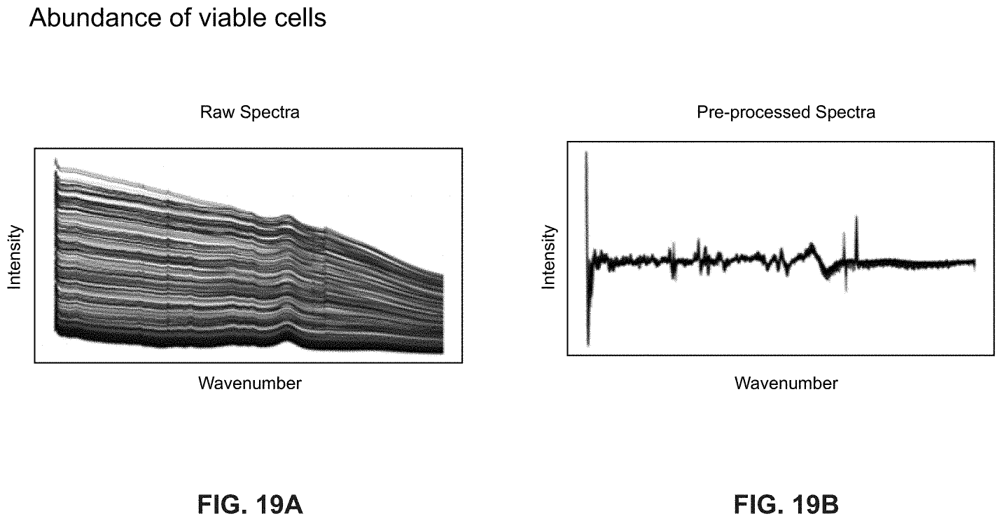

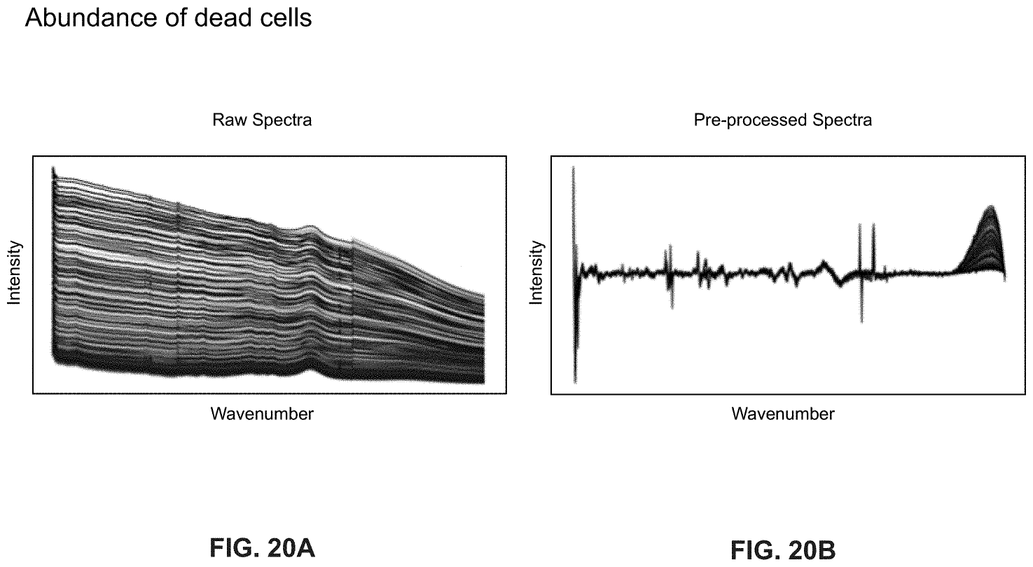

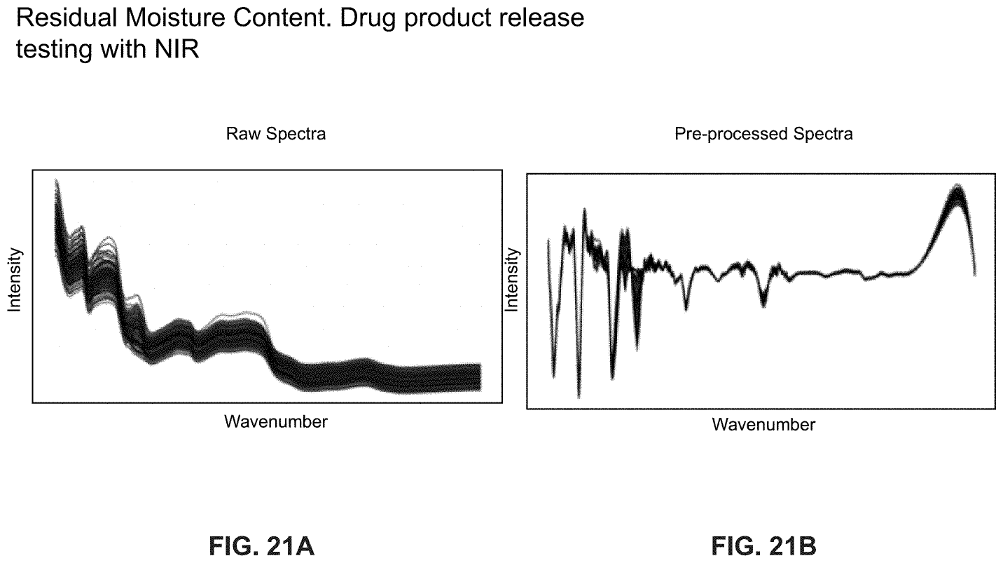

FIGS. 16A-21B show exemplary data pertaining to preprocessing raw spectral data to improve signal quality and machine-learning predictions. FIGS. 16, 17, 18, 19, 20 and 21 correspond to label variables, types of monitoring and processing pipelines corresponding to FIGS. 7, 10, 12, 13, 14 and 15, respectively. For each of the plots, the ranges of x and y coordinates are scaled (e.g., between 0 and 1) relative to a proportion of maximum values observed. Each “A” plot shows a set of input Raman spectra. Each “B” plot shows a corresponding set of pre-processed spectra generated by applying (but not limited to) techniques disclosed herein in accordance with a corresponding processing pipeline. Notably, the particular applied technique(s) for each variable type is different, as it is determined based on the particular spectra depicted in the “A” plots.

It can be seen that, across figures, the spectral preprocessing results in reduced variability across spectra at many, but not all frequencies. It is possible that the frequencies at which cross-spectra variability remains are informative in terms of a particular value of the label variable, while frequencies for which cross-spectra variability is removed are not informative in this regard.

### N. Example 14—Manipulating Raw Spectra Characteristics with Feature Selection

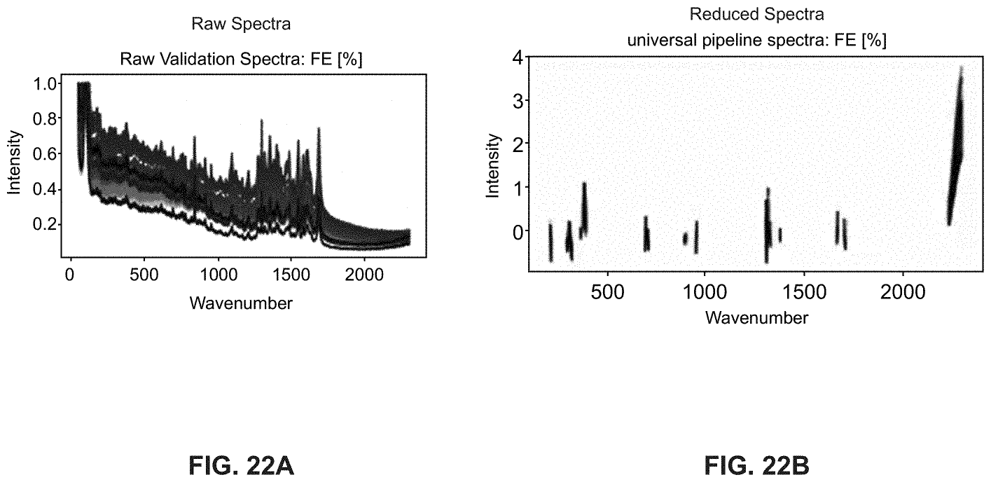

FIGS. 22A-22B show exemplary data pertaining to preprocessing raw spectral data to improve signal quality and machine-learning predictions. The raw input spectra shown in FIG. 22A wavenumbers between 0 and 2000 (e.g., x axis) and a range of y that is scaled (e.g., between 0 and 1) relative to a proportion of maximum values observed. FIG. 22B shows a corresponding set of spectra after a feature-selection process has been performed (e.g., as described in FIGS. 1-3) The feature-selection process was performed in a stage of the processing pipeline (e.g., after pre-processing and before being input into a machine-learning model or before an estimation or prediction of the characteristic is generated).

As demonstrated in FIG. 22B, the set of spectra after the feature-selection process of FIGS. 1-3 was performed is reduced. Wavenumbers that do not contribute to the variability of the wavenumbers were removed from the input spectra as the absence of these wavenumbers either did not or had a marginal effect on the accuracy of the machine-learning model to estimate or predict a characteristic. As shown, only a portion of the wavenumbers of FIG. 22A contribute to the variability and were selected during the feature-selection process.

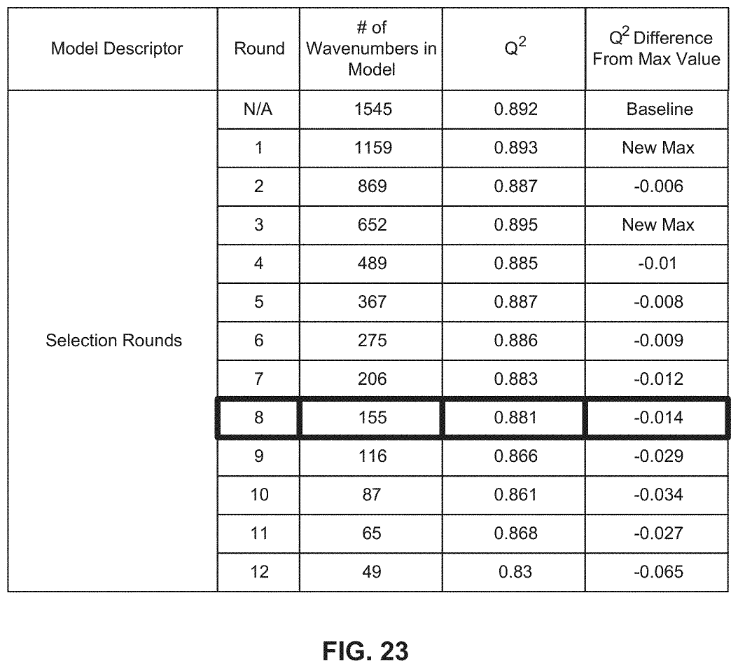

FIG. 23 shows an example execution a feature-selection process that identified a particular reduced set of features for estimating a characteristic of a sample. Each wavenumber was assigned a rank (e.g., as described in FIGS. 1-3). The feature-selection process included 12 iterations with each iteration removing a fixed quantity of wavenumbers and corresponding intensities (e.g., 25%) from the wavenumbers included in the previous iteration. The a threshold deviation of 0.02 was selected to identify the particular iteration having a desirable selection of a wavenumbers. Before the first iteration, there were 1545 wavenumbers). A cross-validation coefficient of the full set of wavenumbers was 0.0892 (e.g., derived according to the process described in FIG. 2), which corresponded to a baseline cross-validation coefficient which subsequent iterations would be compared to.

During iteration 1, the bottom 25% of features (based on the assigned rank) were removed leaving 1159 features. A cross-validation coefficient was derived for the reduced features, which was higher (e.g., by 0.001) than the baseline cross-validation coefficient. As a result, the cross-validation coefficient of iteration 1 became the new baseline cross-validation coefficient. During iteration 2, the bottom 25% of the remaining features (e.g., 25% of the 1159 features from iteration 1) were removed and a cross-validation coefficient of 0.887 was derived for the reduced features.

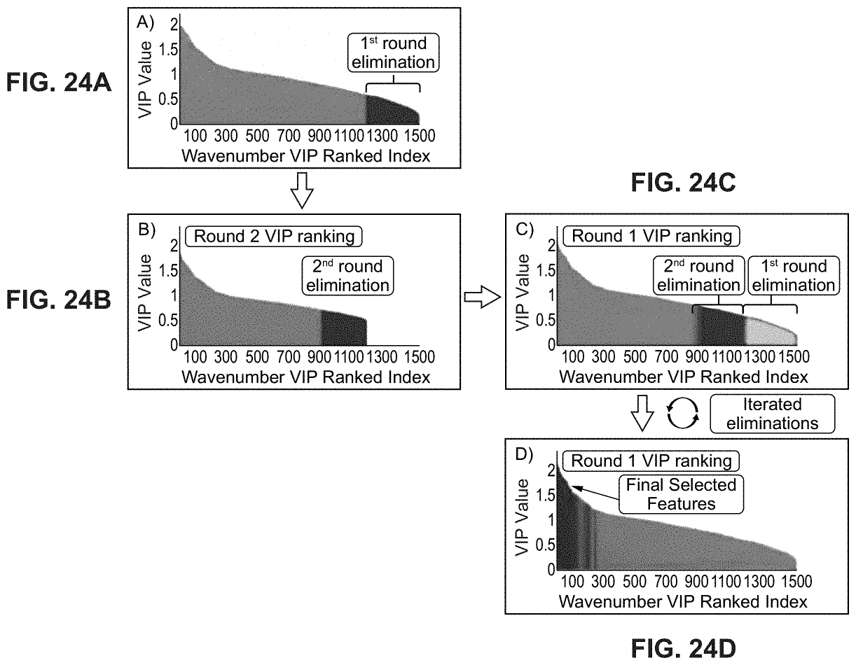

For example, turning to FIG. 24A-24D illustrating a graphical representation of the feature-selection process described in FIGS. 1-3. FIG. 24A illustrates a graph of wavenumbers ordered according to assigned ranks during the first iteration of the example of FIG. 23. As shown in FIG. 24A, the bottom 25% of the wavenumbers were identified for removal from the graph. FIG. 24B illustrates a graph of wavenumbers ordered according to the assigned ranks during a second iterations of the example of FIG. 23. During the second iteration, the bottom 25% of wavenumbers identified from the first iteration were removed. The bottom 25% of the remaining wavenumbers were marked for removal. FIG. 24C illustrates another graph of wavenumbers ordered according to assigned ranks during the second iteration of the example of FIG. 22. AS shown in FIG. 24C, the wavenumbers that removed include the bottom 25% of wavenumbers identified in the first iteration and the bottom 25% of wavenumbers identified in FIG. 24B.

Returning to FIG. 23, at iteration 8 the cross-validation coefficient was 0.881 which was 0.014 from the baseline cross-validation coefficient (e.g., which was updated again during iteration 3 to 0.895). During the next iteration the cross-validation coefficient was 0.866, which was 0.029 from the baseline cross-validation coefficient and exceeded the threshold of 0.020. Iteration 8 was selected to be the particular iteration due to the cross-validation coefficient of iteration 8 being closest to the threshold 0.020 without exceeding the threshold. As a result, the features of iteration 8 were selected for use in generating a predicted characteristic of the sample.

FIG. 24D illustrates a graph of wavenumbers ordered according to assigned ranks during the eighth iteration of the example of FIG. 23. The graph of FIG. 24D distinguishes the wavenumbers the were selected according to the feature-selection process (e.g., as identified by the eighth iteration) from the wavenumbers that were omitted during previous iterations. As shown, a fraction of the full set of wavenumbers were selected.

### V. Exemplary Embodiments

A1. A computer-implemented method comprising:
accessing a data set including a plurality of data elements, each of the data elements including:
   a spectrum generated based on an interaction between one of a plurality of samples and energy from an energy source; and
   a known characteristic of the sample;
initializing a population of candidate solutions, wherein each of the candidate solutions is defined by a set of properties that include:
   an indication that a particular type of pre-processing is to be performed;
   a parameter of a pre-processing to be performed;
   an identification of a type of machine-learning model that is to be used; and/or
   a machine-learning model hyperparameter;
filtering the population of candidate solutions by:
   determining, for each of the candidate solutions and for each of the data elements, a predicted sample characteristic by processing the spectrum of the data element with the set of properties;
   generating, for each of the population of candidate solutions, a fitness metric based on the predicted sample characteristics and the known characteristic of the data elements; and
   selecting an incomplete subset of the population of candidate solutions based on the fitness metrics;
performing one or more additional generation iterations by:
   updating the population of candidate solutions to include a next-generation population of solutions identified using the incomplete subset of the population of candidate solutions and one or more genetic operators; and
   repeating the filtering of the population of candidate solutions using the updated population of candidate solutions; and
generating a processing pipeline based on the set of properties of a particular candidate solution in the incomplete subset of the population of candidate solutions selected during a last generation iteration of the additional generation iterations.

A2. The computer-implemented method of claim A1, further comprising:
accessing another spectrum corresponding to another sample;
generating a predicted characteristic of the other sample by processing the other spectrum in accordance with the processing pipeline; and
outputting the predicted characteristic of the other sample.

A3. The computer-implemented method of any of claims A1-2, wherein, for each data element of the plurality of data elements, the spectrum includes a Raman spectrum or an infrared spectrum.

A4. The computer-implemented method of any of claims A1-A3, wherein the set of properties for the particular candidate solution includes a hyperparameter for a particular type of machine-learning model, the particular type of machine-learning model including:

partial least squares;

random forest; or

support vector machine.

A5. The computer-implemented method of any of claims A1-A4, wherein the set of properties for the particular candidate solution includes a selection of or a hyperparameter for a particular type of machine-learning model, the particular type of machine-learning model being configured to generate classification outputs or numeric outputs.

A6. The computer-implemented method of any of claims A1-A5, wherein the other sample includes large molecules.

A7. The computer-implemented method of any of claims A1-A6, wherein the other sample includes small molecules.

A8. The computer-implemented method of any of claims A1-A7, wherein the predicted characteristic of the other characterizes:

a concentration of one or more small-molecule analytes;

a solvent;

a prevalence of one or more protein variants; or

a protein higher-order structure;

large molecule impurities.

A9. The computer-implemented method of any of claims A1-A8, wherein the processing pipeline includes performing an asymmetric least squares technique to reduce or remove a baseline, and wherein the set of properties for the particular candidate solution includes at least one parameter for the asymmetric least squares technique.

A10. The computer-implemented method of any of claims A1-A9, wherein the processing pipeline includes performing an smoothing technique to reduce or remove a baseline, and wherein the set of properties for the particular candidate solution includes at least one parameter for the smoothing technique.

A11. The computer-implemented method of any of claims A1-A10, wherein, for at least one sample of the plurality of samples, the plurality of data elements includes multiple data elements corresponding to the sample, the multiple data elements including different replicate spectrum generated using the sample.

A12. The computer-implemented method of any of claims A1-A11, further comprising:
partitioning the plurality of data elements into a training subset of the plurality of data elements and a testing subset of the plurality of data elements;
wherein the at least some of the plurality of data elements for which the predicted sample characteristics are determined are defined as the testing subset of the plurality of data elements; and
wherein filtering the population of candidate solutions further includes:
   learning one or more parameters using the testing subset of the plurality of data elements.

A13. The computer-implemented method of any of claims A1-A12, wherein each of the plurality of samples corresponds to a same target chemical structure and to a same target formulation, wherein the plurality of samples includes multiple lot-specific subsets, each of the multiple lot-specific subsets including multiple samples manufactured during an individual lot, and wherein the partitioning of the plurality of data elements includes:

partitioning the individual lots into the training subset and the testing subset; and

partitioning the plurality of data elements based on the lot partitioning.

A14. A computer-implemented method comprising:
collecting the other spectrum for the other sample using an imaging device;
computationally availing the other spectrum to a computer system performing the computer-implemented method of any of claims A1-A13;
receiving, from the computer system, the predicted characteristic;
determining, based on the predicted characteristic, whether a quality-control condition is satisfied;
when the quality control condition is satisfied, distributing the other sample to be administered to a subject; and
when the quality control condition is not satisfied, inhibiting distribution of the other sample for subject administration.

A15. The computer-implemented method of any of claims A1-A14, further comprising:
when the quality control condition is not satisfied, dynamically adjusting one or more parameters associated with production of the other sample.

A16. A computer-implemented method comprising:
providing the other sample for collection of the other spectrum;
computationally availing the other spectrum to a computer system performing the computer-implemented method of any of claims A11-A15;
receiving, from the computer system, the predicted characteristic;
determining, based on the predicted characteristic, whether a quality-control condition is satisfied; and
when the quality control condition is satisfied, initiating or completing one or more a manufacture process configured to manufacture additional samples; and
when the quality control condition is not satisfied, terminating or modifying the one or manufacture process.

A17. A computer-implemented method comprising:
accessing, at a client device, a particular spectrum generated based an interaction between a particular sample and energy from an energy source;
sending, from the client device to a remote computing system, a request for an predicted characteristic of the particular sample to be generated by processing the particular spectrum using a processing pipeline, wherein the processing pipeline was defined by:
   accessing a data set that includes a plurality of data elements corresponding to a plurality of samples, the particular sample being different than each of the plurality of samples, and each data element of the plurality of data elements including:
      a spectrum associated with a sample of the plurality of samples; and
      a known characteristic of the sample;
   initializing a population of candidate solutions, wherein each of the population of candidate solutions is defined by a set of properties that include:
      whether a particular type of pre-processing is to be performed;
      a parameter of a pre-processing to be performed;
      which type of machine-learning model is to be used; and/or
      a machine-learning model hyperparameter;
   filtering the population of candidate solutions by:
      determining, for each of the population of candidate solutions and for each of at least some of the plurality of data elements, a predicted sample characteristic by processing the spectrum of the data element in accordance with the set of properties;
      generating, for each of the population of candidate solutions, a fitness metric based on the predicted sample characteristics and the known characteristics of the at least some of the plurality of data elements; and
      selecting an incomplete subset of the population of candidate solutions based on the fitness metrics;
   performing one or more additional generation iterations by:
      updating the population of candidate solutions to include a next-generation population of solutions identified using the selected incomplete subset of the population of candidate solutions and one or more genetic operators; and
      repeating the filtering of the population of candidate solutions using the updated population of candidate solutions; and
   defining a processing pipeline based on the set of properties of a particular candidate solution in the incomplete subset of the population of candidate solutions selected during a last generation iteration of the one or more additional generation iterations; and
receiving, at the client device and from the remote computing system, the predicted characteristic of the particular sample.

A18. The computer-implemented method of any of claims A1-A17, further comprising:
collecting the particular spectrum using spectroscopy to initiate emission of the energy from the energy source.

A19. The computer-implemented method of any of claims A1-A18, further comprising:
modifying the processing pipeline to include performing a feature-selection process, that selects, from a set of intensities of the spectrum, one or more intensities for use in generating the predicted characteristic of the predicted sample, wherein the feature-selection processing is performed prior to generation of the predicted characteristic by the processing pipeline.

A20. The computer-implemented method of any of claims A1-A19, wherein the feature-selection process includes:
identifying, from the spectrum, a set of wavenumbers, each wavenumber being associated with an intensity value;
defining a score for each wavenumber of the set of wavenumbers using a regression analysis;
sorting the set of wavenumbers according to the score of each wavenumber of the set of wavenumbers;
performing one or more feature-selection iterations, wherein each feature-selection iteration includes:
   generating a subset of the set of wavenumbers by removing one or more wavenumbers of the spectrum having a lowest score; and
   generating a model-validation score based on a cross-validation of the subset of the set of wavenumbers on the machine-learning model;
   selecting, from the one or more feature-selection iterations, a particular feature-selection iteration of the one or more feature-selection iterations that includes a model-validation score that is closest to a threshold; and
   selecting, for use in generating the predicted characteristic by the processing pipeline, intensities that correspond to the subset of the set of wavenumbers of the particular feature-selection iteration.

A21. A system comprising:
one or more data processors; and
a non-transitory computer readable storage medium containing instructions which, when executed on the one or more data processors, cause the one or more data processors to perform part or all of one or more methods disclosed herein.

A22. A computer-program product tangibly embodied in a non-transitory machine-readable storage medium, including instructions configured to cause one or more data processors to perform part or all of one or more methods disclosed herein.

### V. Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The present description provides preferred exemplary embodiments only, and is not intended to limit the scope, applicability or configuration of the disclosure. Rather, the present description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the present description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

1. A computer-implemented method comprising:
accessing a data set including a plurality of data elements, each of the data elements including:
a spectrum generated based on an interaction between one sample of a plurality of samples and energy from an energy source; and
a known characteristic of the sample;
initializing a population of candidate solutions, wherein each of the candidate solutions is defined by a set of properties that include:
an indication that a particular type of pre-processing is to be performed;
a parameter of a pre-processing to be performed;
an identification of a type of machine-learning model that is to be used; and/or
a machine-learning model hyperparameter;
filtering the population of candidate solutions by:
determining, for each of the candidate solutions and for each of the data elements, a predicted sample characteristic by processing the spectrum of the data element with the set of properties;
generating, for each of the population of candidate solutions, a fitness metric based on the predicted sample characteristics and the known characteristic of the data elements; and
selecting an incomplete subset of the population of candidate solutions based on the fitness metrics;
performing one or more additional generation iterations by:
updating the population of candidate solutions to include a next-generation population of solutions identified using the incomplete subset of the population of candidate solutions and one or more genetic operators; and
repeating the filtering of the population of candidate solutions using the updated population of candidate solutions; and
generating a processing pipeline based on the set of properties of a particular candidate solution in the incomplete subset of the population of candidate solutions selected during a last generation iteration of the additional generation iterations.

2. The computer-implemented method of claim 1, further comprising:
accessing another spectrum corresponding to another sample;
generating a predicted characteristic of the other sample by processing the other spectrum in accordance with the processing pipeline; and
outputting the predicted characteristic of the other sample.

3. The computer-implemented method of claim 1, wherein, for each data element of the plurality of data elements, the spectrum includes a Raman spectrum or an infrared spectrum.

4. The computer-implemented method of claim 1, wherein the set of properties for the particular candidate solution includes a hyperparameter for a particular type of machine-learning model, the particular type of machine-learning model including:
partial least squares;
random forest; or
support vector machine.

5. The computer-implemented method of claim 1, wherein the set of properties for the particular candidate solution includes a selection of or a hyperparameter for a particular type of machine-learning model, the particular type of machine-learning model being configured to generate classification outputs or numeric outputs.

6. The computer-implemented method of claim 1, wherein the other sample includes large molecules.

7. The computer-implemented method of claim 1, wherein the other sample includes small molecules.

8. The computer-implemented method of claim 1, wherein the predicted characteristic of the other sample characterizes:
a concentration of one or more small-molecule analytes;
a solvent;
a prevalence of one or more protein variants;
a protein higher-order structure; or
large molecule impurities.

9. The computer-implemented method of claim 1, wherein the processing pipeline includes performing an asymmetric least squares technique to reduce or remove a baseline, and wherein the set of properties for the particular candidate solution includes at least one parameter for the asymmetric least squares technique.

10. The computer-implemented method of claim 1, wherein the processing pipeline includes performing an smoothing technique to reduce or remove a baseline, and wherein the set of properties for the particular candidate solution includes at least one parameter for the smoothing technique.

11. The computer-implemented method of claim 1, wherein, for at least one sample of the plurality of samples, the plurality of data elements includes multiple data elements corresponding to the sample, the multiple data elements including different replicate spectrum generated using the sample.

12. The computer-implemented method of claim 1, further comprising:
partitioning the plurality of data elements into a training subset of the plurality of data elements and a testing subset of the plurality of data elements;
wherein the at least some of the plurality of data elements for which the predicted sample characteristics are determined are defined as the testing subset of the plurality of data elements; and
wherein filtering the population of candidate solutions further includes:
learning one or more parameters using the testing subset of the plurality of data elements.

13. The computer-implemented method of claim 12, wherein each of the plurality of samples corresponds to a same target chemical structure and to a same target formulation, wherein the plurality of samples includes multiple lot-specific subsets, each of the multiple lot-specific subsets including multiple samples manufactured during an individual lot, and wherein the partitioning of the plurality of data elements includes:
partitioning the individual lots into the training subset and the testing subset; and
partitioning the plurality of data elements based on the lot partitioning.

14. The computer-implemented method of claim 1, further comprising:
accessing another spectrum corresponding to another sample;
generating a predicted characteristic of the other sample by processing the other spectrum with the processing pipeline;
determining, based on the predicted characteristic, whether a quality-control condition is satisfied;
when the quality control condition is satisfied, distributing the other sample to be administered to a subject; and
when the quality control condition is not satisfied, inhibiting distribution of the other sample for subject administration.

15. The computer-implemented method of claim 14, further comprising:
when the quality control condition is not satisfied, dynamically adjusting one or more parameters associated with production of the other sample.

16. The computer-implemented method of claim 1, further comprising:
performing a feature-selection process that selects, from a set of intensities of the spectrum, one or more intensities for use in generating the predicted characteristic of the predicted sample, wherein the feature-selection processing is performed prior to generation of the predicted characteristic by the processing pipeline.

17. The computer-implemented method of claim 16, wherein the feature-selection process includes:
identifying, from the spectrum, a set of wavenumbers, each wavenumber being associated with an intensity value;
defining a score for each wavenumber of the set of wavenumbers using a regression analysis;
sorting the set of wavenumbers according to the score of each wavenumber of the set of wavenumbers;
performing one or more feature-selection iterations, wherein each feature-selection iteration includes:
generating a subset of the set of wavenumbers by removing one or more wavenumbers of the spectrum having a lowest score; and
generating a model-validation score based on a cross-validation of the subset of the set of wavenumbers on the machine-learning model;
selecting, from the one or more feature-selection iterations, a particular feature-selection iteration of the one or more feature-selection iterations that includes a model-validation score that is closest to a threshold; and
selecting, for use in generating the predicted characteristic by the processing pipeline, intensities that correspond to the subset of the set of wavenumbers of the particular feature-selection iteration.

18. The computer-implemented method of claim 1, further comprising:
accessing another spectrum corresponding to another sample;
generating a predicted characteristic of the other sample by processing the other spectrum in accordance with the processing pipeline;
receiving the predicted characteristic;
determining, based on the predicted characteristic, whether a quality-control condition is satisfied; and
when the quality control condition is satisfied, initiating or completing one or more a manufacture process configured to manufacture additional samples; and
when the quality control condition is not satisfied, terminating or modifying the one or manufacture process.

19. A system comprising:
one or more data processors; and
a non-transitory computer readable storage medium containing instructions which, when executed on the one or more data processors, cause the one or more data processors to perform a set of actions that include:
accessing a data set including a plurality of data elements, each of the data elements including:
a spectrum generated based on an interaction between one sample of a plurality of samples and energy from an energy source; and
a known characteristic of the sample;
initializing a population of candidate solutions, wherein each of the candidate solutions is defined by a set of properties that include:
an indication that a particular type of pre-processing is to be performed;
a parameter of a pre-processing to be performed;
an identification of a type of machine-learning model that is to be used; and/or
a machine-learning model hyperparameter;
filtering the population of candidate solutions by:
determining, for each of the candidate solutions and for each of the data elements, a predicted sample characteristic by processing the spectrum of the data element with the set of properties;
generating, for each of the population of candidate solutions, a fitness metric based on the predicted sample characteristics and the known characteristic of the data elements; and
selecting an incomplete subset of the population of candidate solutions based on the fitness metrics;
performing one or more additional generation iterations by:
updating the population of candidate solutions to include a next-generation population of solutions identified using the incomplete subset of the population of candidate solutions and one or more genetic operators; and
repeating the filtering of the population of candidate solutions using the updated population of candidate solutions; and
generating a processing pipeline based on the set of properties of a particular candidate solution in the incomplete subset of the population of candidate solutions selected during a last generation iteration of the additional generation iterations.

20. A computer-program product tangibly embodied in a non-transitory machine-readable storage medium, including instructions configured to cause one or more data processors to perform a set of actions that include:
accessing a data set including a plurality of data elements, each of the data elements including:
a spectrum generated based on an interaction between one sample of a plurality of samples and energy from an energy source; and
a known characteristic of the sample;
initializing a population of candidate solutions, wherein each of the candidate solutions is defined by a set of properties that include:
an indication that a particular type of pre-processing is to be performed;
a parameter of a pre-processing to be performed;
an identification of a type of machine-learning model that is to be used; and/or
a machine-learning model hyperparameter;
filtering the population of candidate solutions by:
determining, for each of the candidate solutions and for each of the data elements, a predicted sample characteristic by processing the spectrum of the data element with the set of properties;
generating, for each of the population of candidate solutions, a fitness metric based on the predicted sample characteristics and the known characteristic of the data elements; and
selecting an incomplete subset of the population of candidate solutions based on the fitness metrics;
performing one or more additional generation iterations by:
updating the population of candidate solutions to include a next-generation population of solutions identified using the incomplete subset of the population of candidate solutions and one or more genetic operators; and
repeating the filtering of the population of candidate solutions using the updated population of candidate solutions; and
generating a processing pipeline based on the set of properties of a particular candidate solution in the incomplete subset of the population of candidate solutions selected during a last generation iteration of the additional generation iterations.
